(19) United States
(12) Patent Application Publication
(10) Pub. No.: US 2006/0057696 A1
(43) Pub. Date: Mar. 16, 2006

(54) Sugar chain synthases

(76) Inventors: Shou Takashima, Tokyo (JP); Masafumi Tsujimoto, Saitama (JP); Shuichi Tsuji, Kanagawa (JP)

Correspondence Address: GREENBLUM & BERNSTEIN, P.L.C., 1950 ROLAND CLARKE PLACE, RESTON, VA, 20191, US

(21) Appl. No.: 10/501,930

(22) PCT Filed: Jan. 30, 2003
(86) PCT No.: PCT/JP03/00883

§ 371 Date: Mar. 17, 2005

(30) Foreign Application Priority Data
Jan. 30, 2002 (JP) 2002-21159
Apr. 24, 2002 (JP) 2002-122673

(51) Int. Cl. C12N 9/10; C12P 21/06

(52) U.S. Cl. 435/193; 435/69.1; 435/320.1; 435/325

(57) ABSTRACT

The present invention provides O-glycan α2,8-sialyltransferase which has novel substrate specificity and substrate selectivity, and β-galactoside α2,6-sialyltransferase which has novel action and substrate specificity. The sialyltransferase of the present invention can be used as a medicament for suppression of cancer metastasis, prevention of virus infection, suppression of inflammatory response, or activation of neural cells.

### TECHNICAL FIELD

The present invention relates to a glycosylating enzyme and DNA encoding the enzyme. More specifically, the present invention relates to an enzyme (O-glycan α2,8-sialyltransferase, ST8Sia VI) that efficiently transfers sialic acid through an α2,8 linkage onto the sialic acid portion of a sugar chain having a Sia α2,3(6)Gal (Sia: sialic acid; Gal: galactose) structure at the terminus of O-glycans such as mucin, and DNA encoding the above enzyme; and an enzyme (ST6Gal II) that efficiently transfers sialic acid through an α2,6 linkage onto the galactose portion of a sugar chain having a Galβ1,4GlcNAc (Gal: galactose; GluNAc: N-acetylglucosamine) structure at the terminus of sugar chains such as oligosaccharide, and DNA encoding the above enzyme. The O-glycan α2,8-sialyltransferase and β-galactoside α2,6-sialyltransferase of the present invention are useful as a medicament having effects of suppression of cancer metastasis, prevention of virus infection, suppression of inflammatory response or activation of neural cells, as a reagent for increasing physiological action by adding sialic acid to a sugar chain, or as an enzyme inhibitor.

### BACKGROUND ART

Sialic acid is a substance responsible for important physiological actions such as cell-cell communication, cell-substrate interaction, and cell adhesion. The presence of sialic acid-containing sugar chains has been known, and some of such chains are expressed in stage-specific manner during development and differentiation, or in tissue-specific manner. Sialic acid exists at the terminal position of the sugar chain of a glycoprotein or glycolipid. Introduction of sialic acid into these sites is carried out emzymatically by transfer of sialic acid portion from CMP-Sia.

Enzymes having a function in such enzymatic introduction of sialic acid (sialic acid tranfer) belong to a member of glycosyltransferases called sialyltransferases. So far, 18 types of sialyltransferases have been known with regard to mammals. These sialyltransferases are broadly divided into 4 families (Tsuji, S. (1996) J. Biochem. 120, 1-13). This is to say, these 4 families are: α2,3-sialyltransferase (ST3Gal-family) that transfers sialic acid onto galactose through an α2,3 linkage; α2,6-sialyltransferase (ST6Gal-family) that transfers sialic acid onto galactose through an α2,6 linkage; GalNAc α2,6-sialyltransferase (ST6GalNAc-family) that transfers sialic acid onto N-acetylgalactosamine through an α2,6 linkage; and α2,8-sialyltransferase (ST8Sia-family) that transfers sialic acid onto sialic acid through an α2,8 linkage.

Of these, with regard to α2,8-sialyltransferase, cDNA cloning of 5 types of the enzymes (ST8Sia I-V) have been achieved so far, and their enzymatic properties have been elucidated (Yamamoto, A. et al. (1996) J. Neurochem. 66, 26-34; Kojima, N. et al. (1995) FEBS Lett. 360, 1-4; Yoshida, Y. et al. (1995) J. Biol. Chem. 270, 14628-14633; Yoshida, Y. et al. (1995) J. Biochem. 118, 658-664; Kono, M. et al. (1996) J. Biol. Chem. 271, 29366-29371). ST8Sia I is an enzyme for synthesizing a ganglioside GD3, and ST8Sia V is also an enzyme for synthesizing gangliosides GD1c, GT1a, GQ1b, GT3, and so on. ST8Sia II and IV are enzymes for synthesizing polysialic acid on the N-glycans of a neural cell adhesion molecule (NCAM). ST8Sia III is an enzyme for transferring sialic acid onto Siaα2,3Galβ1,4GlcNAc structures found in the N-glycans of glycoproteins and glycolipids. The preferred substrates for all of these enzymes are glycolipids or N-glycans. There have been only two reports in which these enzymes exhibit activity toward O-glycans. A case where ST8Sia II and IV synthesize oligosialic acid/polysialic acid on O-glycans found in an isoform of NCAM, and a case where ST8Sia III acts on the O-glycans of an adipocyte-specific glycoprotein AdipoQ (Suzuki, M. et al. (2000) Glycobiology 10, 1113; and Sato C, et al. (2001) J. Biol. Chem. 276, 28849-28856). Thus, the previously reported α2,8-sialyltransferases do not generally utilize O-glycans as preferred substrates. The existence of α2,8-sialyltransferase which utilizes such an O-glycans as preferred substrates has been unknown.

Moreover, so far, cDNA cloning of only one type of β-galactoside α2,6-sialyltransferase (ST6Gal I) has been achieved, and its enzymatic properties have been elucidated (Hamamoto, T. and Tsuji, S. (2001) ST6Gal-I in Handbook of Glycosyltransferases and Related Genes (Taniguchi, N. et al. Eds.) pp. 295-300). ST6Gal I shows its activity on glycoproteins, oligosaccharides, and gangliosides, which have a Galβ1,4GlcNAc structure at the terminal position of their carbohydrates. ST6Gal I is an enzyme having broad substrate specificity, whose substrate can be not only the Galβ1,4GlcNAc structure, but also lactose (Galβ1,4Glc), or a Galβ1,3GlcNAc structure in some cases. If a functional oligosaccharide is synthesized using an enzyme having wide substrate specificity such as ST6Gal I, there is a possibility that by-products might be generated when there are impurities in the raw materials, as these impurities would also serve as substrates. To solve this problem, an enzyme having high selectivity is required in terms of substrate specificity. However, so far, the enzyme having β-galactoside α2,6-sialyltransferase activity with high selectivity in terms of substrate specificity has not been identified from mammals.

### DISCLOSURE OF THE INVENTION

As stated above, only 5 types of α2,8-sialyltransferases have been known so far. Main substrates for all of these enzymes are glycoproteins having N-glycans or glycolipids such as gangliosides. These enzymes show no activity toward glycoproteins having O-glycans, or show only a limited activity. It is the first object of the present invention to provide a novel O-glycan α2,8-sialyltransferase showing high activity toward O-glycans. It is also the object of the present invention to clone the cDNA encoding O-glycan α2,8-sialyltransferase, so as to provide a DNA sequence encoding the above O-glycan α2,8-sialyltransferase and an amino acid sequence of the above enzyme. Moreover, it is also the object of the present invention to allow a portion necessary for the activity of the above O-glycan α2,8-sialyltransferase to express as a protein in a large quantity.

Furthermore, as stated above, only one type of β-galactoside α2,6-sialyltransferase (ST6Gal I) has been known in mammals. This enzyme shows activity toward glycoproteins, oligosaccharides, or gangliosides, which have a Galβ1,4GlcNAc structure at the terminal position of their carbohydrates. ST6Gal I is an enzyme having a wide substrate specificity, whose substrate can be not only the Galβ1,4GlcNAc structure, but also lactose (Galβ1,4Glc), or a Galβ1,3GlcNAc structure in some cases. It is the second object of the present invention to provide a novel β-galactoside α2,6-sialyltransferase, which solves the above problem regarding broad substrate specificity and shows highly selective substrate specificity to a Galβ1,4GlcNAc structure on oligosaccharide, and DNA encoding the enzyme.

The present inventors have made intensive studies to achieve the above-described objects. The present inventors have screened mouse brain and heart cDNA libraries, and have also performed PCR using cDNA derived from mouse kidney as a template, so that they have succeeded in cloning the cDNA encoding O-glycan α2,8-sialyltransferase. Moreover, using the amino acid sequence of human sialyltransferase ST6Gal I, the present inventors have searched the expressed sequence tag (dbEST) database for a clone encoding a novel sialyltransferase showing a homology with the above enzyme, and have obtained the EST clones of GenBank™ accession Nos. BE613250, BE612797, and BF038052. Furthermore, using the information on these nucleotide sequences, the present inventors have searched both the dbEST database and the database of high throughput genomic sequences of the human genome, and have obtained information on the nucleotide sequences of the related EST clones and the genome sequence of this gene. Based on the above obtained nucleotide sequence information, primers for the polymerase chain reaction method (PCR) were prepared, and PCR was carried out using human colon-derived cDNA as a template. The obtained amplified fragment was ligated to the DNA fragment derived from the above-obtained EST clone, so as to obtain a clone encoding the entire coding region. Thereafter, it was confirmed that a protein encoded by the above clone has the activity of β-galactoside α2,6-sialyltransferase. The present invention has been completed based on these findings.

That is to say, the present invention provides O-glycan α2,8-sialyltransferase, which is characterized in that it has the following substrate specificity and substrate selectivity.

Substrate specificity: the substrates of the enzyme are glycoconjugates having a Siaα2,3(6)Gal structure (wherein Sia represents sialic acid and Gal represents galactose) at the terminus thereof.

Substrate selectivity: the enzyme incorporates sialic acids into O-glycans more preferentially than into glycolipids or N-glycans.

Preferably, the present invention provides O-glycan α2,8-sialyltransferase having either one of the following amino acid sequences: (1) an amino acid sequence shown in SEQ ID NO: 1 or 3; or (2) an amino acid sequence comprising a deletion, substitution, and/or addition of one or several amino acids with respect to the amino acid sequence shown in SEQ ID NO: 1 or 3, and having O-glycan α2,8-sialyltransferase activity.

In another aspect of the present invention, the O-glycan α2,8-sialyltransferase gene encoding the above-described amino acid sequence of the O-glycan α2,8-sialyltransferase of the present invention is provided.

Preferably, the present invention provides the O-glycan α2,8-sialyltransferase gene having any one of the following nucleotide sequences: (1) a nucleotide sequence corresponding to a portion between nucleotide 77 and nucleotide 1270 of a nucleotide sequence shown in SEQ ID NO: 2; (2) a nucleotide sequence comprising a deletion, substitution, and/or addition of one or several nucleotides with respect to the nucleotide sequence corresponding to a portion between nucleotide 77 and nucleotide 1270 of the nucleotide sequence shown in SEQ ID NO: 2, and encoding a protein having O-glycan α2,8-sialyltransferase activity; (3) a nucleotide sequence corresponding to a portion between nucleotide 92 and nucleotide 1285 of a nucleotide sequence shown in SEQ ID NO: 4; and (4) a nucleotide sequence comprising a deletion, substitution, and/or addition of one or several nucleotides with respect to the nucleotide sequence corresponding to a portion between nucleotide 92 and nucleotide 1285 of the nucleotide sequence shown in SEQ ID NO: 4, and encoding a protein having O-glycan α2,8-sialyltransferase activity.

In another aspect of the present invention, the followings are provided: a recombinant vector (preferably, an expression vector) comprising the above-described O-glycan α2,8-sialyltransferase gene of the present invention; a transformant transformed with the above recombinant vector; and a method for producing the enzyme of the present invention wherein the above transformant is cultured and the enzyme of the present invention is collected from the culture.

In another aspect of the present invention, a protein which comprises an active domain of O-glycan α2,8-sialyltransferase having any one of the following amino acid sequences is provided: (1) an amino acid sequence corresponding to a portion between positions 26 and 398 of the amino acid sequence shown in SEQ ID NO: 1; (2) an amino acid sequence comprising a deletion, substitution, and/or addition of one or several amino acids with respect to the amino acid sequence corresponding to a portion between positions 26 and 398 of the amino acid sequence shown in SEQ ID NO: 1, and having O-glycan α2,8-sialyltransferase activity; (3) an amino acid sequence corresponding to a portion between positions 68 and 398 of the amino acid sequence shown in SEQ ID NO: 3; and (4) an amino acid sequence comprising a deletion, substitution, and/or addition of one or several amino acids with respect to the amino acid sequence corresponding to a portion between positions 68 and 398 of the amino acid sequence shown in SEQ ID NO: 3, and having O-glycan α2,8-sialyltransferase activity.

In another aspect of the present invention, an extracellular secretory protein is provided, which comprises a polypeptide portion of the active domain and a signal peptide of the O-glycan α2,8-sialyltransferase of the present invention, and has O-glycan α2,8-sialyltransferase activity.

In another aspect of the present invention, a gene encoding the above-described extracellular secretory protein of the present invention is provided.

In another aspect of the present invention, the followings are provided: a recombinant vector (preferably, an expression vector) comprising a gene encoding the above-described extracellular secretory protein of the present invention; a transformant transformed with the above recombinant vector; and a method for producing the protein of the present invention wherein the above transformant is cultured and the enzyme of the present invention is collected from the culture.

In another aspect of the present invention, a β-galactoside α2,6-sialyltransferase, which is characterized in that it has the following action and substrate specificity, is provided.

(1) Action;

The enzyme transfers sialic acid through an α2,6 linkage into the galactose portion of a sugar chain having a galactose β1,4N-acetylglucosamine structure at the terminus thereof.

(2) Substrate Specificity

The substrate of the enzyme is a sugar chain having a galactose β1,4N-acetylglucosamine structure at the terminus thereof, and lactose and a sugar chain having a galactose β1,3N-acetylglucosamine structure at the terminus thereof are not the substrate of the enzyme.

In another aspect of the present invention, a β-galactoside α2,6-sialyltransferase having either one of the following amino acid is provided: (1) an amino acid sequence shown in SEQ ID NO: 5 or 7; or (2) an amino acid sequence comprising a deletion, substitution, and/or addition of one or several amino acids with respect to the amino acid sequence shown in SEQ ID NO: 5 or 7, and having β-galactoside α2,6-sialyltransferase activity.

In another aspect of the present invention, a β-galactoside α2,6-sialyltransferase gene encoding the above-described amino acid sequence of the β-galactoside α2,6-sialyltransferase of the present invention is provided.

In another aspect of the present invention, a β-galactoside α2,6-sialyltransferase gene having any one of the following nucleotide sequences is provided: (1) a nucleotide sequence corresponding to a portion between nucleotide 176 and nucleotide 1762 of a nucleotide sequence shown in SEQ ID NO: 6; (2) a nucleotide sequence comprising a deletion, substitution, and/or addition of one or several nucleotides with respect to the nucleotide sequence corresponding to a portion between nucleotide 176 and nucleotide 1762 of the nucleotide sequence shown in SEQ ID NO: 6, and encoding a protein having β-galactoside α2,6-sialyltransferase activity; (3) a nucleotide sequence corresponding to a portion between nucleotide 3 and nucleotide 1574 of a nucleotide sequence shown in SEQ ID NO: 8; and (4) a nucleotide sequence comprising a deletion, substitution, and/or addition of one or several nucleotides with respect to the nucleotide sequence corresponding to a portion between nucleotide 3 and nucleotide 1574 of the nucleotide sequence shown in SEQ ID NO: 8, and encoding a protein having β-galactoside α2,6-sialyltransferase activity.

In another aspect of the present invention, a recombinant vector comprising the β-galactoside α2,6-sialyltransferase gene of the present invention is provided.

The recombinant vector of the present invention is preferably an expression vector.

In another aspect of the present invention, a transformant transformed with the recombinant vector of the present invention is provided.

In another aspect of the present invention, a method for producing the enzyme of the present invention is provided, wherein the transformant of the present invention is cultured and the enzyme of the present invention is collected from the culture.

In another aspect of the present invention, a protein comprising an active domain of β-galactoside α2,6-sialyltransferase having any one of the following amino acid sequences is provided: (1) an amino acid sequence corresponding to a portion between positions 33 and 529 of the amino acid sequence shown in SEQ ID NO: 5; (2) an amino acid sequence comprising a deletion, substitution, and/or addition of one or several amino acids with respect to the amino acid sequence corresponding to a portion between positions 33 and 529 of the amino acid sequence shown in SEQ ID NO: 5, and having β-galactoside α2,6-sialyltransferase activity; (3) an amino acid sequence corresponding to a portion between positions 31 and 524 of the amino acid sequence shown in SEQ ID NO: 7; and (4) an amino acid sequence comprising a deletion, substitution, and/or addition of one or several amino acids with respect to the amino acid sequence corresponding to a portion between positions 31 and 524 of the amino acid sequence shown in SEQ ID NO: 7, and having β-galactoside α2,6-sialyltransferase activity.

In another aspect of the present invention, an extracellular secretory protein is provided, which comprises a polypeptide portion of the active domain and a signal peptide of the β-galactoside α2,6-sialyltransferase of the present invention, and has β-galactoside α2,6-sialyltransferase activity.

In another aspect of the present invention, a gene encoding the above-described protein of the present invention is provided.

In another aspect of the present invention, a recombinant vector comprising the above-described gene of the present invention is provided.

The recombinant vector of the present invention is preferably an expression vector.

In another aspect of the present invention, a transformant transformed with the recombinant vector of the present invention is provided.

In another aspect of the present invention, a method for producing the protein of the present invention is provided, wherein the transformant of the present invention is cultured and the protein of the present invention is collected from the culture.

### BRIEF DESCRIPTION OF THE DRAWINGS

FIG. 1 shows the nucleotide sequences of ST8Sia VI cDNA of a mouse and a human, and the deduced amino acid sequences. A transmembrane domain is underlined, sialyl motif L is double-underlined, and sialyl motif S is dashed-underlined. Histidine and glutamic acid, which are conserved in sialyl motif VS, are boxed. Asparagine residues of the potential N-linked glycosylation sites are overlined. FIG. 1A shows mouse ST8Sia VI, and FIG. 1B shows human ST8Sia VI.

FIG. 2 shows a comparison of amino acid sequences.

FIG. 2A shows a comparison made among the amino acid sequences of mouse sialyltransferases ST8Sia I, ST8Sia V, and ST8Sia VI. The conserved amino acid residues among these sialyltransferases are boxed. Sialyl motif L is duble-underlined, and sialyl motif S is dashed-underlined. The conserved histidine and glutamic acid residues in sialyl motif VS are marked with asterisks.

FIG. 2B shows a comparison made between the amino acid sequence of mouse (m) ST8Sia VI and that of human (h) ST8Sia VI. Amino acids conserved between both the enzymes are boxed.

FIG. 3 shows an analysis of linkage specificity. A, [¹⁴C]-NeuAc-incorporated GM3 sialylated by the secretory recombinant protein PA-mST8Sia VI of mouse ST8Sia VI was treated with α2,3-, and α2,6-linkage specific sialidase (NANase II) or with α2,3-, α2,6-, α2,8-, and α2,9-linkage specific sialidase (NANase III), and then the reaction products were analyzed by HPTLC (where a developing solvent consists of chloroform:methanol: 0.02% CaCl₂=55:45:10) (upper panel). [¹⁴C]-NeuAc-incorporated 3′-sialyllactose sialylated by the secretory recombinant protein PA-hST8Sia VI of human ST8Sia VI was treated with NANase II or NANase III, and then the reaction products were analyzed by HPTLC (where a developing solvent consists of 1-propanol:ammonia water:water=6:1:2.5) (lower panel). B, GM3 was sialylated by PA-mST8Sia VI, and the reaction product was analyzed by TLC immunostaining (lower panel). Lane 1, GD3 (1 μg); lane 2, GM3 (1 μg); and lane 3, the reaction product. The reaction product was reacted with an anti-GD3 monoclonal antibody KM641 and peroxidase-conjugated anti-mouse IgG+IgM (H+L) antibody, and then detected using an ECL system.

In FIG. 4, Fetuin was [¹⁴C]-NeuAc-incorporated by ST8Sia III or ST8Sia VI and then treated with N-glycanase. The [¹⁴C]-NeuAc-incorporated Fetuin was treated with N-glycanase, and the treated product was analyzed by SDS-PAGE. Thereafter, it was visualized with a BAS2000 radio image analyzer.

FIG. 5 shows effects of the overexpression of the mouse ST8Sia VI full-length cDNA in COS-7 cells.

FIG. 5A shows results of the TLC immunostaining using an anti-NeuAcα2,8NeuAcα2,3Gal antibody S2-566. Lane 1, standard GD3 substance (0.5 μg); lane 2, standard GQ1b (0.5 μg); lane 3, an acidic glycolipid fraction extracted from control COS-7 cells (30 mg); and lane 4, an acidic glycolipid fraction extracted from COS-7 cells (30 mg) into which a mouse full-length ST8Sia VI expression vector pRc/CMV-ST8Sia VI had been introduced.

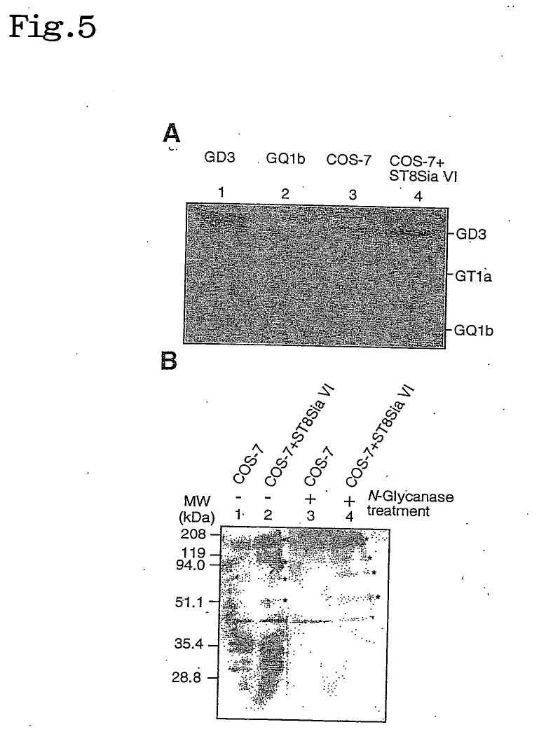

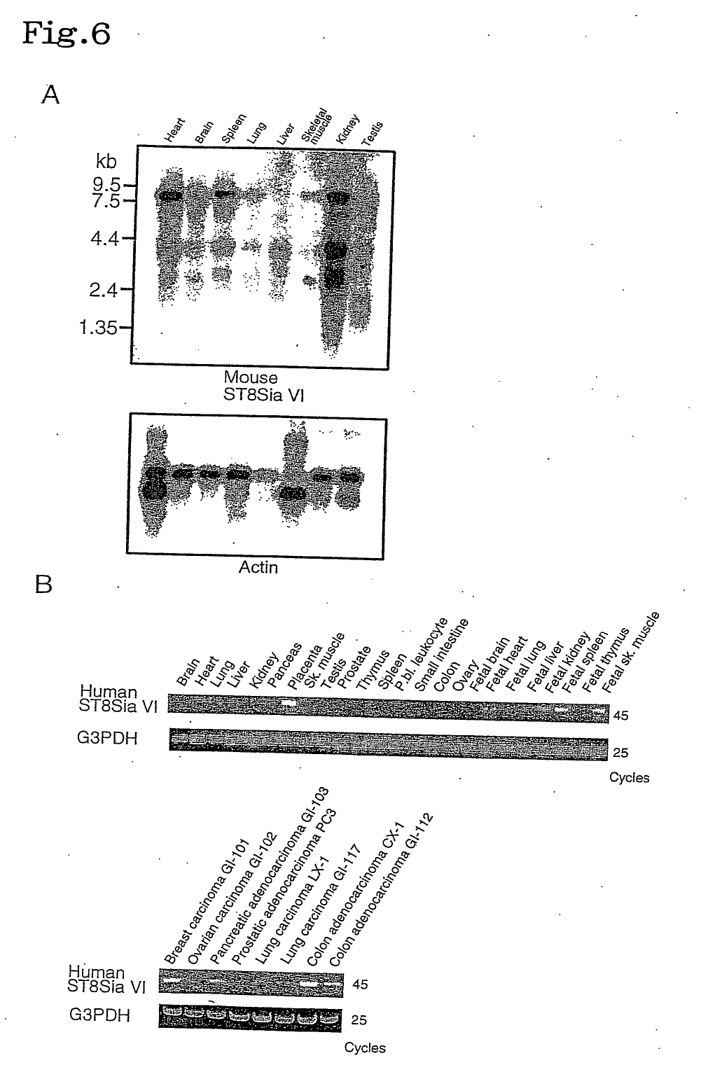

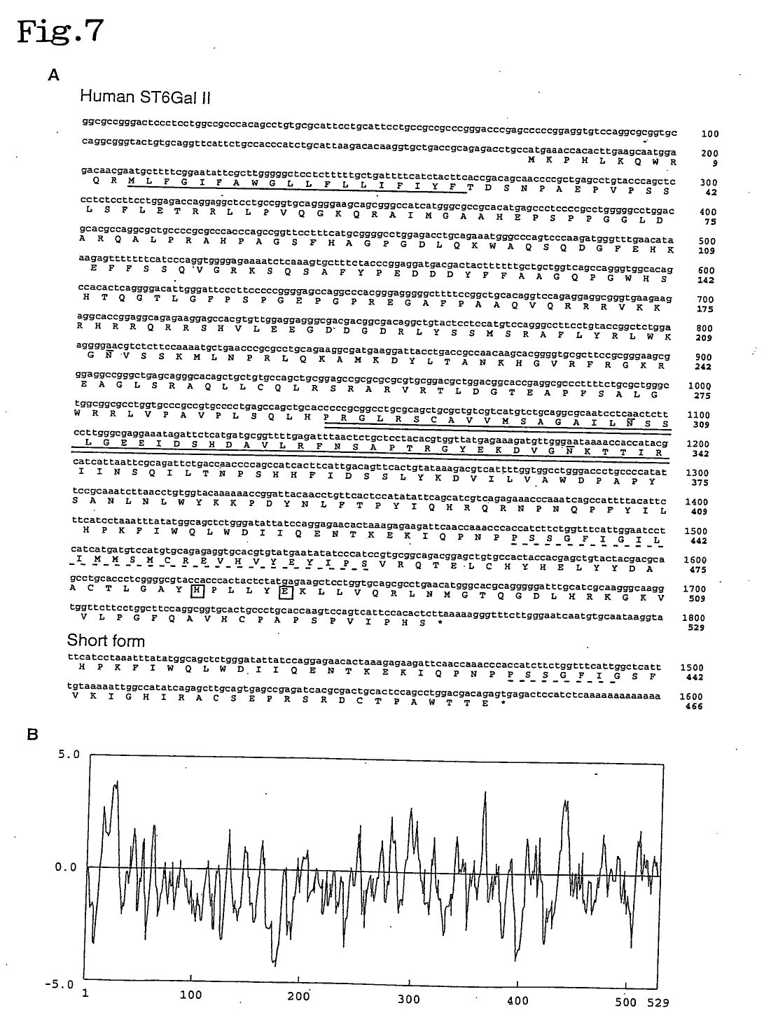

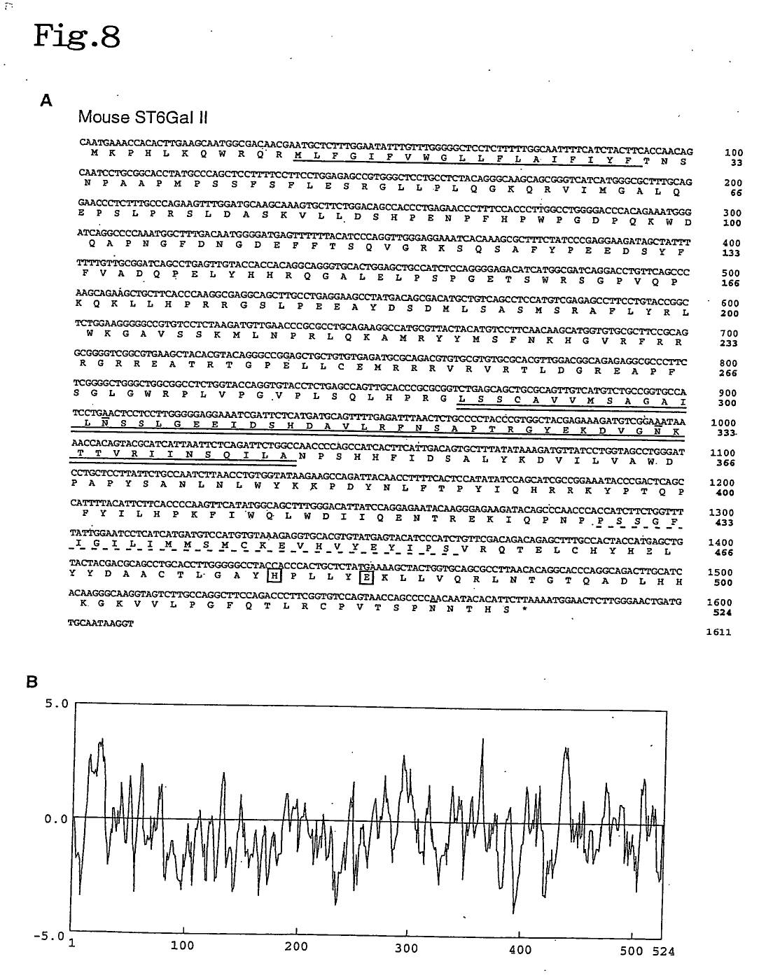

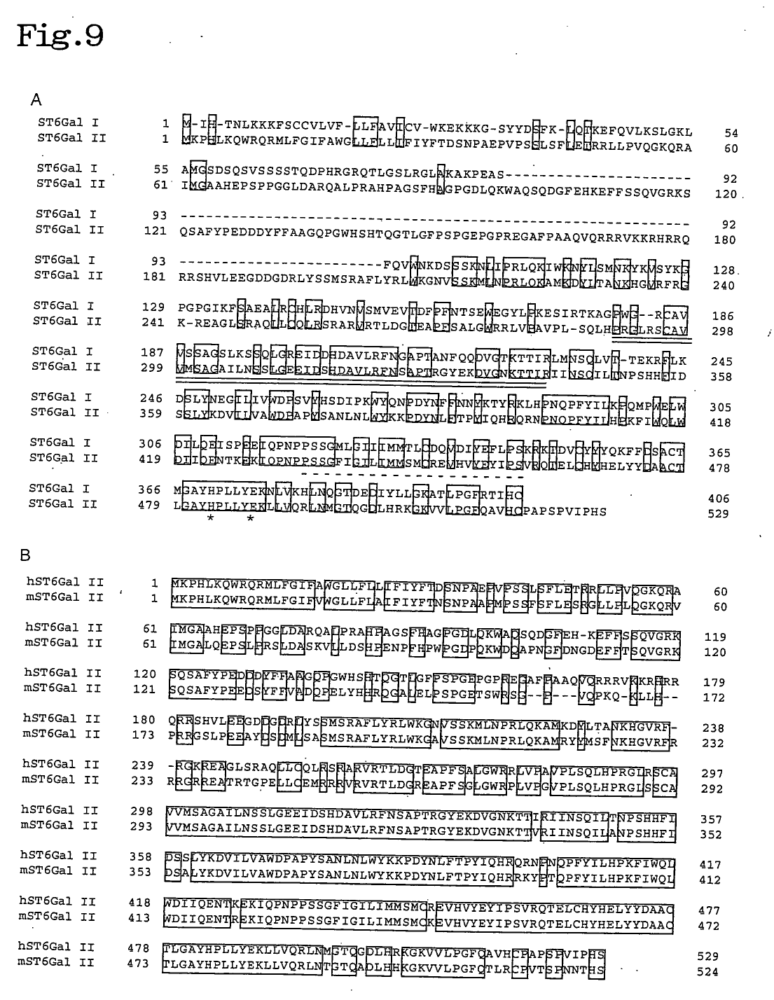

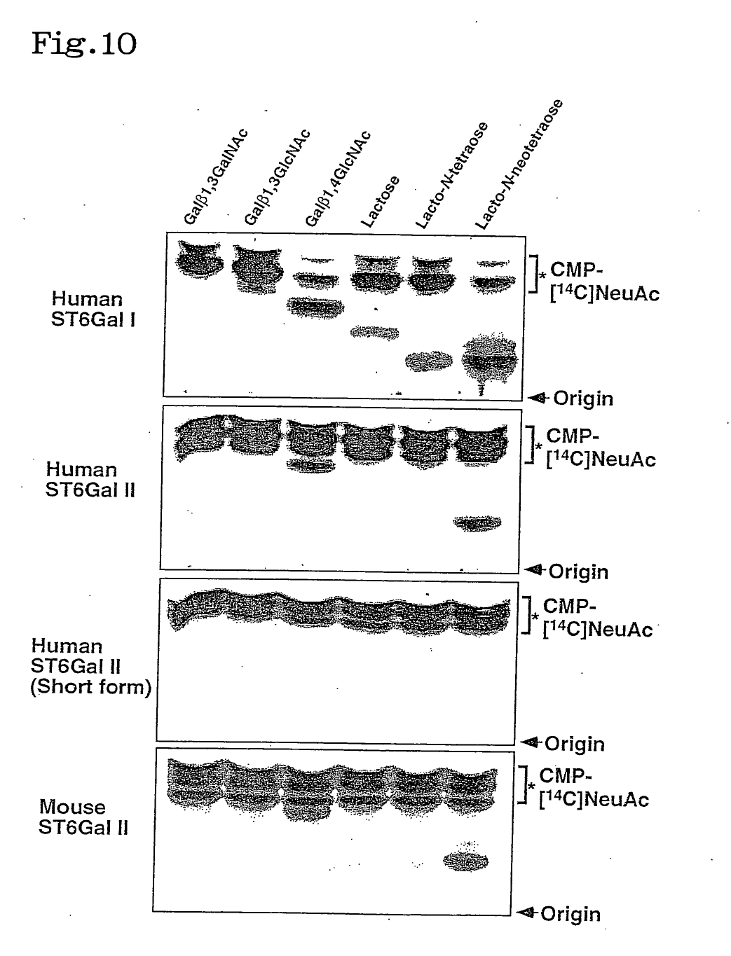

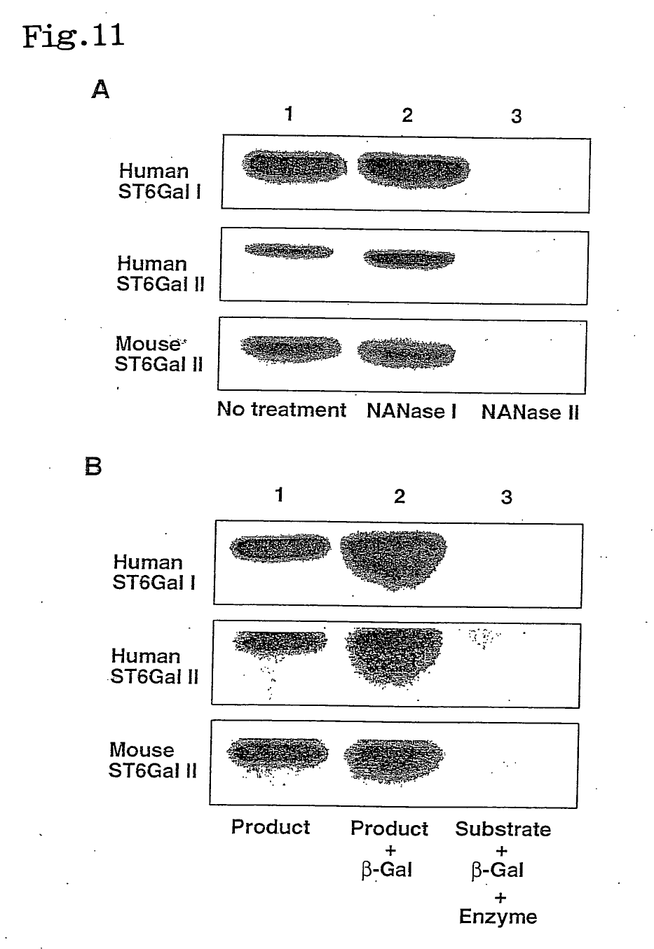

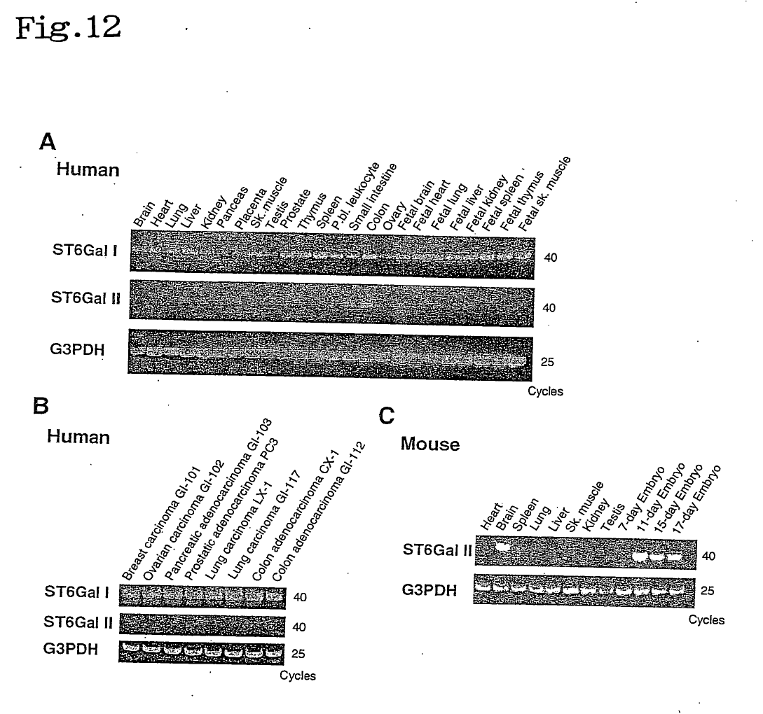

In FIG. 5B, microsome fractions were prepared from COS-7 cells, or COS-7 cells into which pRc/CMV-ST8Sia VI has been introduced. Then they were subjected to SDS-PAGE (45 μg/lane), and transferred to a PVDF membrane, and western blotting was performed using an S2-566 antibody. Lane 1, the microsome fraction prepared from control COS-7 cells; lane 2, the microsome fraction prepared from COS-7 cells into which pRc/CMV-ST8Sia VI has been introduced; lane 3, the N-glycanase-treated microsome fraction prepared from the control COS-7 cells; and lane 4, the N-glycanase-treated microsome fraction prepared from the COS-7 cells into which pRc/CMV-ST8Sia VI had been introduced. Asterisks are attached to main bands which are recognized by the S2-566 antibody and are generated as a result of the introduction of ST8Sia VI cDNA.

FIG. 6 shows the expression patterns of mouse and human ST8Sia VI genes.

FIG. 6A shows results of the expression pattern of the mouse ST8Sia VI gene analyzed by northern blotting with poly(A)+RNA (approximately 2 μg/lane) prepared from various types of mouse organs.

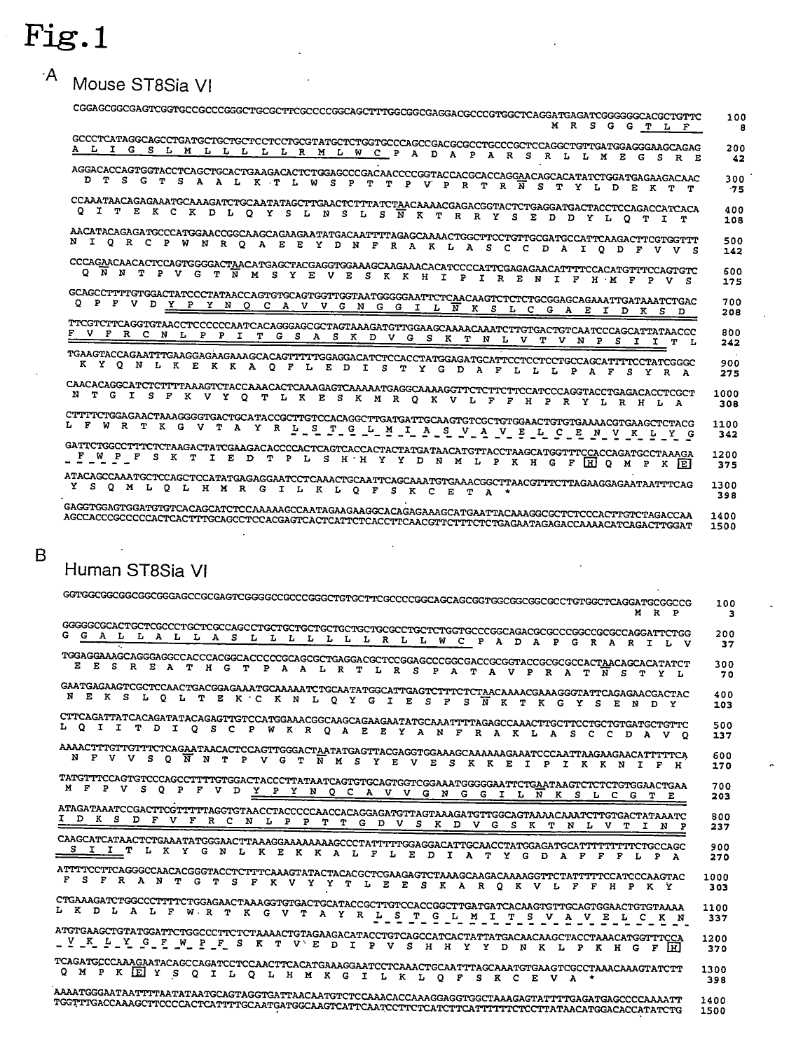

FIG. 6B shows results of the expression pattern of the human ST8Sia VI gene analyzed by PCR using a Multiple Tissue cDNA Panel (Clontech). As human ST8Sia VI-specific primers, 5′-CCAGTGTCCCAGCCTTTTGT-3′ (corresponding to nucleotides 608-627 in FIG. 1B) (SEQ ID NO: 17) and 5′-TGAGTGGGGAAGCTTTGGTC-3′ (corresponding to a complementary strand of nucleotides 1407-1426 in FIG. 1B) (SEQ ID NO: 18) were used. The size of the PCR amplified fragment is 819 bp.

FIG. 7 shows the nucleotide sequence of human ST6Gal II cDNA, its deduced amino acid sequence, and the hydropathy plot of the protein.

FIG. 7A shows the nucleotide sequence of human ST6Gal II cDNA, and its deduced amino acid sequence. The transmembrane domain is underlined. Sialyl motif L is double underlined, and sialyl motif S is dashed underlined. Histidine and glutamic acid, which are conserved in sialyl motif VS, are boxed. Asparagine residues of the potential N-linked glycosilation sites are overlined.

FIG. 7B shows the hydropathy plot of human ST6Gal II. A large hydrophobic region on the N-terminal region is predicted to be a transmembrane domain.

FIG. 8 shows the nucleotide sequence of mouse ST6Gal II cDNA, its deduced amino acid sequence, and the hydropathy plot of the protein.

FIG. 8A shows the nucleotide sequence of mouse ST6Gal II cDNA and its deduced amino acid sequence. The transmembrane domain is underlined. Sialyl motif L is double underlined, and sialyl motif S is dashed underlined. Histidine and glutamic acid, which are conserved in sialyl motif VS, are boxed. Asparagine residues of the potential N-linked glycosylation sites are overlined.

FIG. 8B shows the hydropathy plot of mouse ST6Gal II. A large hydrophobic region on the N-terminal region is predicted to be a transmembrane domain.

FIG. 9 shows a comparison of amino acid sequences.

FIG. 9A shows a comparison of the amino acid sequence of human sialyltransferase ST6Gal I and that of human sialyltransferase ST6Gal II. The conserved amino acid residues between these enzymes are boxed. Sialyl motif L is double underlined, and sialyl motif S is dashed underlined. The conserved histidine and glutamic acid residues in sialyl motif VS are marked with asterisks.

FIG. 9B shows a comparison of the amino acid sequence of human (h) ST6Gal II and that of mouse (m) ST6Gal II. The conserved amino acid residues between these enzymes are boxed.

FIG. 10 shows the activity toward oligosaccharides. The enzyme reaction was carried out using various oligosaccharides as substrates (10 μg/lane). The figure shows the reaction product analyzed by HPTLC (where a developing solvent consists of 1-propanol:ammonia water:water=6:1:2.5).

FIG. 11 shows an analysis of linkage specificity. A, [¹⁴C]-NeuAc-incorporated Galβ1,4GlcNAc sialylated by human ST6Gal I (upper panel), human ST6Gal II (middle panel), and mouse ST6Gal II (lower panel) (lane 1) was treated with α2,3-linkage specific sialidase (NANase I, lane 2) or with α2,3-, and α2,6-linkage specific sialidase (NANase II, lane 3), and then the reaction products were analyzed by HPTLC (where a developing solvent consists of 1-propanol:ammonia water:water=6:1:2.5). B, [¹⁴C]-NeuAc-incorporated Galβ1,4GlcNAc sialylated by human ST6Gal I (upper panel), human ST6Gal II (middle panel), and mouse ST6Gal II (lower panel) (lane 1) was treated with β-galactosidase (lane 2). As a control, Galβ1,4GlcNAc was treated with β-galactosidase, and then an enzyme reaction was performed (lane 3). These were analyzed by HPTLC (where a developing solvent consists of 1-propanol:ammonia water:water=6:1:2.5). The broad bands in lane 2 were caused by the effects of high concentration of ammonium sulfate in the β-galactosidase solution.

FIG. 12 shows analysis of the expression patterns of human ST6Gal I and ST6Gal II genes, and a mouse ST6Gal II gene. Using human ST6Gal I- and ST6Gal II-specific primers and a Multiple tissue cDNA panel (Clontech) of human tissues (A) or human tumor cells (B), the expression patterns of both genes were analyzed by PCR. One PCR cycle consists of 94° C. for 1 minute, 50° C. for 1 minute, and 72° C. for 1.5 minutes. 25 cycles of PCR was performed for the glyceraldehyde 3-phosphate dehydrogenase (G3PDH) gene, and 40 cycles of PCR was performed for human ST6Gal I and ST6Gal II genes. The reaction products were analyzed by agarose gel electrophoresis. “Sk. Muscle” means skeletal muscle, and “P. bl. Leukocyte” means peripheral blood leukocyte. FIG. 12C shows the expression pattern of the mouse ST6Gal II gene analyzed by PCR using mouse ST6Gal II-specific primers and the Multiple tissue cDNA panel (Clontech) of mouse tissues.

### BEST MODE FOR CARRYING OUT THE INVENTION

The embodiments of the present invention and the methods for carrying out the present invention will be described in detail below.

### (1) Enzyme and Protein of the Present Invention

The O-glycan α2,8-sialyltransferase of the present invention is characterized in that it has the following substrate specificity and substrate selectivity.

Substrate specificity: the substrates of the enzyme are glycoconjugates having a Siaα2,3(6)Gal structure (wherein Sia represents sialic acid and Gal represents galactose) at the terminus thereof.

Substrate selectivity: the enzyme incorporates sialic acids into O-glycan more preferentially than into glycolipids or N-glycans.

The above-described substrate specificity and substrate selectivity are characteristics which have been demonstrated by mouse- and human-derived O-glycan α2,8-sialyltransferases obtained in examples described in the present specification. The O-glycan α2,8-sialyltransferase of the present invention is not only derived from a mouse and a human, and it is easily understandable for a person skilled in the art that the same type of O-glycan α2,8-sialyltransferase exists in the tissues of other mammals and that those O-glycan α2,8-sialyltransferases have a high homology to one another.

Such O-glycan α2,8-sialyltransferases are characterized in that they have the above-described substrate specificity and substrate selectivity. These enzymes are also included in the scope of the present invention.

Examples of such an O-glycan α2,8-sialyltransferase may include natural enzymes derived from mammalian tissues and mutants thereof, and extracellular secretory proteins catalyzing the transfer of sialic acid to O-glycans through an α2,8-linkage, which are produced by genetic recombination, such as those produced in examples described later. These are also included in the scope of the present invention.

O-glycan α2,8-sialyltransferase having either one of the following amino acid sequences may be one example of the O-glycan α2,8-sialyltransferase of the present invention: (1) an amino acid sequence shown in SEQ ID NO: 1 or 3; or (2) an amino acid sequence comprising a deletion, substitution, and/or addition of one or several amino acids with respect to the amino acid sequence shown in SEQ ID NO: 1 or 3, and having O-glycan α2,8-sialyltransferase activity.

In addition, it is to be understood that an active domain of the O-glycan α2,8-sialyltransferase of the present invention and proteins having O-glycan α2,8-sialyltransferase activity obtained by alteration or modification of a portion of the amino acid sequence thereof are all included in the scope of the present invention. Preferred examples of such an active domain may include an active domain of O-glycan α2,8-sialyltransferase corresponding to a portion between positions 26 and 398 of the amino acid sequence shown in SEQ ID NO: 1 and an active domain of O-glycan α2,8-sialyltransferase corresponding to a portion between positions 68 and 398 of the amino acid sequence shown in SEQ ID NO: 3. A sequence portion between positions 26 and approximately 100 of the amino acid sequence shown in SEQ ID NO: 1 or 3 is a region called stem, and it is considered that this region is not necessarily required for the activity. Accordingly, a region corresponding to positions 101 to 398 of the amino acid sequence shown in SEQ ID NO: 1 or 3 may be used as an active domain of O-glycan α2,8-sialyltransferase.

That is to say, the present invention provides a protein which comprises an active domain of O-glycan α2,8-sialyltransferase having any one of the following amino acid sequences: (1) an amino acid sequence corresponding to a portion between positions 26 and 398 of the amino acid sequence shown in SEQ ID NO: 1; (2) an amino acid sequence comprising a deletion, substitution, and/or addition of one or several amino acids with respect to the amino acid sequence corresponding to a portion between positions 26 and 398 of the amino acid sequence shown in SEQ ID NO: 1, and having O-glycan α2,8-sialyltransferase activity. (3) an amino acid sequence corresponding to a portion between positions 68 and 398 of the amino acid sequence shown in SEQ ID NO: 3; and (4) an amino acid sequence comprising a deletion, substitution, and/or addition of one or several amino acids with respect to the amino acid sequence corresponding to a portion between positions 68 and 398 of the amino acid sequence shown in SEQ ID NO: 3, and having O-glycan α2,8-sialyltransferase activity.

On the other hand, the β-galactoside α2,6-sialyltransferase of the present invention is characterized in that it has the following action and substrate specificity.

(1) Action

The enzyme transfers sialic acid through an α2,6 linkage into the galactose portion of a sugar chain having a galactose β1,4N-acetylglucosamine structure at the terminus thereof.

(2) Substrate Specificity

The substrate of the enzyme is a sugar chain having a galactose β1,4N-acetylglucosamine structure at the terminus thereof, and lactose and a sugar chain having a galactose β1,3N-acetylglucosamine structure at the terminus thereof are not the substrate of the enzyme.

The above-described action and substrate specifity are characteristics which have been demonstrated by mouse- and human-derived β-galactoside α2,6-sialyltransferases obtained in examples described in the present specification. The β-galactoside α2,6-sialyltransferase of the present invention is not only derived from a mouse and a human, but it is easily understood for a person skilled in the art that the same type of β-galactoside α2,6-sialyltransferase exists in the tissues of other mammals and that those β-galactoside α2,6-sialyltransferases have a high homology to one another.

Such β-galactoside α2,6-sialyltransferases are characterized in that they have the above-described action and substrate specifity. These enzymes are also included in the scope of the present invention.

Examples of such a β-galactoside α2,6-sialyltransferase may include natural enzymes derived from mammalian tissues and mutants thereof, and extracellular secretory proteins catalyzing the transfer of sialic acid to β-galactosides through an α2,6-linkage, which are produced by genetic recombination. These are also included in the scope of the present invention.

β-galactoside α2,6-sialyltransferase having either one of the following amino acid sequences may be one example of the β-galactoside α2,6-sialyltransferase of the present invention: (1) an amino acid sequence shown in SEQ ID NO: 5 or 7; or (2) an amino acid sequence comprising a deletion, substitution, and/or addition of one or several amino acids with respect to the amino acid sequence shown in SEQ ID NO: 5 or 7, and having β-galactoside α2,6-sialyltransferase activity.

In addition, it is to be understood that an active domain of the β-galactoside α2,6-sialyltransferase of the present invention and proteins having β-galactoside α2,6-sialyltransferase activity obtained by alteration or modification of a portion of the amino acid sequence thereof are all included in the scope of the present invention. A preferred example of such an active domain may be an active domain of β-galactoside α2,6-sialyltransferase corresponding to a portion between positions 33 and 529 of the amino acid sequence shown in SEQ ID NO: 5. A sequence portion between positions 31 and approximately 200 of the amino acid sequence shown in SEQ ID NO: 5 is a region called stem, and it is considered that this region is not necessarily required for the activity. Accordingly, a region corresponding to positions 201 to 529 of the amino acid sequence shown in SEQ ID NO: 1 may be used as an active domain of β-galactoside α2,6-sialyltransferase.

Likewise, another preferred example of such an active domain may be an active domain of β-galactoside α2,6-sialyltransferase corresponding to a portion between positions 31 and 524 of the amino acid sequence shown in SEQ ID NO: 7. A sequence portion between positions 31 and approximately 200 of the amino acid sequence shown in SEQ ID NO: 7 is a region called stem, and it is considered that this region is not necessarily required for the activity. Accordingly, a region corresponding to positions * 201 to 524 of the amino acid sequence shown in SEQ ID NO: 7 may be used as an active domain of β-galactoside α2,6-sialyltransferase.

That is to say, the present invention provides a protein which comprises an active domain of β-galactoside α2,6-sialyltransferase having any one of the amino acid sequences described below.

In another aspect of the present invention, a protein which comprises an active domain of β-galactoside α2,6-sialyltransferase having any one of amino acid sequences described below is provided: (1) an amino acid sequence corresponding to a portion between positions 33 and 529 of the amino acid sequence shown in SEQ ID NO: 5; (2) an amino acid sequence comprising a deletion, substitution, and/or addition of one or several amino acids with respect to the amino acid sequence corresponding to a portion between positions 33 and 529 of the amino acid sequence shown in SEQ ID NO: 5, and having β-galactoside α2,6-sialyltransferase activity; (3) an amino acid sequence corresponding to a portion between positions 31 and 524 of the amino acid sequence shown in SEQ ID NO: 7, and (4) an amino acid sequence comprising a deletion, substitution, and/or addition of one or several amino acids with respect to the amino acid sequence corresponding to a portion between positions 31 and 524 of the amino acid sequence shown in SEQ ID NO: 7, and having β-galactoside α2,6-sialyltransferase activity.

In the present specification, the range of “one or several” in the expression “an amino acid sequence comprising a deletion, substitution, and/or addition of one or several amino acids” is not particularly limited. For example, it means 1 to 20 amino acids, preferably 1 to 10 amino acids, more preferably 1 to 7 amino acids, further more preferably 1 to 5 amino acids, and particularly preferably 1 to 3 amino acids.

A method for obtaining the enzyme or protein of the present invention is not particularly limited. The protein of the present invention may be a protein synthesized by chemical synthesis, or recombinant protein produced by genetic recombination.

When a recombinant protein is produced, first, DNA encoding the protein is required to be obtained. Suitable primers are designed based on the information regarding amino acid sequences and nucleotide sequences shown in SEQ ID NOS: 1 to 8 of the sequence listing in the present specification. Thereafter, using the obtained primers, PCR is carried out with a suitable cDNA library as a template, so as to obtain DNA encoding the enzyme of the present invention.

For example, methods for isolating cDNA encoding O-glycan α2,8-sialyltransferases having amino acid sequences shown in SEQ ID NOS: 1 and 3, and cDNA encoding β-galactoside α2,6-sialyltransferases having amino acid sequences shown in SEQ ID NOS: 5 and 7 are described in detail in examples described later. However, a method for isolating cDNA encoding the O-glycan α2,8-sialyltransferase or β-galactoside α2,6-sialyltransferase of the present invention is not limited thereto. A person skilled in the art could easily isolate cDNA of interest by referring to the methods described in examples below and appropriately modifying or altering them.

Moreover, when a partial fragment of DNA encoding the enzyme of the present invention is produced by the above-described PCR, the produced DNA fragments can be successively ligated to one another, so as to obtain DNA encoding a desired enzyme. The obtained DNA can be then introduced into a suitable expression system, so as to generate the enzyme of the present invention. Expression of the enzyme in such an expression system will be described later in the specification.

An extracellular secretory protein, which comprises a polypeptide portion of the active domain of the O-glycan α2,8-sialyltransferase or β-galactoside α2,6-sialyltransferase of the present invention and a signal peptide, and has O-glycan α2,8-sialyltransferase activity or β-galactoside α2,6-sialyltransferase activity is also included in the present invention.

In some cases, the O-glycan α2,8-sialyltransferase and β-galactoside α2,6-sialyltransferase of the present invention may remain in cells after the expression and may not be secreted outside of the cells. In addition, there is a possibility that the production of the enzymes may be decreased when the intracellular concentration thereof exceeds a certain limit. In order to effectively use the activity of the above O-glycan α2,8-sialyltransferase to transfer sialic acid to O-glycans through an α2,8-linkage and the activity of the above β-galactoside β2,6-sialyltransferase to transfer sialic acid to β-galactosides through an α2,6-linkage, a soluble form of proteins retaining the activities of the present enzymes and being secreted from cells during the expression may be produced. An example of such a protein may be an extracellular secretory protein, which comprises a signal peptide and a polypeptide portion of the active domain of O-glycan α2,8-sialyltransferase or β-galactoside α2,6-sialyltransferase which is involved in the activity of the O-glycan α2,8-sialyltransferase or β-galactoside α2,6-sialyltransferase of the present invention, and catalyzes the transfer of sialic acid to O-glycans through an α2,8-linkage or to β-galactosides through an α2,6-linkage. For example, a fusion protein with a signal peptide of mouse immunoglobulin IgM or protein A is preferred embodiments of the secretory protein of the present invention.

Sialyltransferases that have been cloned so far have a domain structure similar to that of other glycosyltransferases. This is to say, the previously cloned sialyltransferases comprise an NH₂-terminal short cytoplasmic tail, a hydrophobic signal anchor domain, a stem region having proteolytic sensitivity, and a COOH-terminal large active domain (Paulson, J. C. and Colley, K. J., J. Biol. Chem., 264, 17615-17618, 1989). In order to examine the position of a transmembrane domain of the O-glycan α2,8-sialyltransferase or β-galactoside α2,6-sialyltransferase of the present invention, a hydropathy plot prepared according to the method of Kyte and Doolittle (Kyte, J. and Doolittle, R. F., J. Mol. Biol., 157, 105-132, 1982) can be used. Moreover, in order to estimate an active domain portion, recombinant plasmids into which various types of fragments are introduced are produced and used. An example of such methods is described in detail, for example, in PCT/JP94/02182. However, a method for confirming the position of a transmembrane domain or estimating an active domain portion is not limited thereto.

In order to produce an extracellular secretory protein which comprises a polypeptide portion of the active domain of O-glycan α2,8-sialyltransferase or β-galactoside α2,6-sialyltransferase and a signal peptide, for example, a sequence corresponding to the active domain of O-glycan α2,8-sialyltransferase or β-galactoside α2,6-sialyltransferase may be subjected to inframe fusion with an immunoglobulin signal peptide sequence as a signal peptide. As such a method, the method of Jobling (Jobling, S. A. and Gehrke, L., Nature (Lond.), 325, 622-625, 1987), for example, can be used. Further, as is described in detail in examples of the present specification, a fusion protein with a signal peptide of mouse immunoglobulin IgM or protein A may also be produced. However, the type of a signal peptide, the method of the fusion of a signal peptide with an active domain, and the method of solubilization are not limited to those described above. A person skilled in the art may appropriately select a polypeptide portion which is an active domain of O-glycan α2,8-sialyltransferase or β-galactoside α2,6-sialyltransferase, and may fuse the selected polypeptide portion with any available signal peptide by a suitable method, so as to produce an extracellular secretory protein.

### (2) Gene of the Present Invention

The present invention provides a gene encoding the amino acid sequence of the O-glycan α2,8-sialyltransferase of the present invention, and a gene encoding the amino acid sequence of the β-galactoside α2,6-sialyltransferase of the present invention.

Specific examples of a gene encoding the amino acid sequence of the O-glycan α2,8-sialyltransferase of the present invention may include genes having any one of the following nucleotide sequences: (1) a nucleotide sequence corresponding to a portion between nucleotide 77 and nucleotide 1270 of a nucleotide sequence shown in SEQ ID NO: 2; (2) a nucleotide sequence comprising a deletion, substitution, and/or addition of one or several nucleotides with respect to the nucleotide sequence corresponding to a portion between nucleotide 77 and nucleotide 1270 of the nucleotide sequence shown in SEQ ID NO: 2, and encoding a protein having O-glycan α2,8-sialyltransferase activity; (3) a nucleotide sequence corresponding to a portion between nucleotide 92 and nucleotide 1285 of a nucleotide sequence shown in SEQ ID NO: 4; and (4) a nucleotide sequence comprising a deletion, substitution, and/or addition of one or several nucleotides with respect to the nucleotide sequence corresponding to a portion c between nucleotide 92 and nucleotide 1285 of the nucleotide sequence shown in SEQ ID NO: 4, and encoding a protein having O-glycan α2,8-sialyltransferase activity.

Specific examples of a gene encoding the amino acid sequence of the β-galactoside α2,6-sialyltransferase of the present invention may include genes having any one of the following nucleotide sequences: (1) a nucleotide sequence corresponding to a portion between nucleotide 176 and nucleotide 1762 of a nucleotide sequence shown in SEQ ID NO: 6; (2) a nucleotide sequence comprising a deletion, substitution, and/or addition of one or several nucleotides with respect to the nucleotide sequence corresponding to a portion between nucleotide 176 and nucleotide 1762 of the nucleotide sequence shown in SEQ ID NO: 6, and encoding a protein having β-galactoside α2,6-sialyltransferase activity; (3) a nucleotide sequence corresponding to a portion between nucleotide 3 and nucleotide 1574 of a nucleotide sequence shown in SEQ ID NO: 8; and (4) a nucleotide sequence comprising a deletion, substitution, and/or addition of one or several nucleotides with respect to the nucleotide sequence corresponding to a portion between nucleotide 3 and nucleotide 1574 of the nucleotide sequence shown in SEQ ID NO: 8, and encoding a protein having β-galactoside α2,6-sialyltransferase activity.

The range of “one or several” in the expression “a nucleotide sequence comprising a deletion, substitution, and/or addition of one or several nucleotides” in the present specification is not particularly limited. For example, it means 1 to 60 nucleotides, preferably 1 to 30 nucleotides, more preferably 1 to 20 nucleotides, further more preferably 1 to 10 nucleotides, further more preferably 1 to 5 nucleotides, and particularly preferably 1 to 3 nucleotides.

A gene encoding a protein comprising an active domain of the O-glycan α2,8-sialyltransferase or β-galactoside α2,6-sialyltransferase of the present invention, and a gene encoding an extracellular secretory protein which comprises a polypeptide portion which is the above active domain and a signal peptide and has O-glycan α2,8-sialyltransferase activity or β-galactoside α2,6-sialyltransferase activity, are also included in the scope of the present invention.

The gene of the present invention can be obtained by the above-described method.

A method of introducing a desired mutation into a certain nucleic acid sequence is known to those skilled in the art. For example, known techniques such as site-directed mutagenesis, PCR using degenerated oligonucleotides, or exposure of cells containing nucleic acid to a mutagenic agent or radioactive ray are used as appropriate, whereby DNA comprising a mutation can be constructed. Such known techniques are described, for example, in Molecular Cloning: A laboratory Manual, 2^{nd} Ed., Cold Spring Harbor Laboratory, Cold Spring Harbor, N.Y., 1989; and Current Protocols in Molecular Biology, Supplements 1 to 38, John Wiley & Sons (1987-1997).

### (3) Recombinant Vector of the Present Invention

The gene of the present invention can be inserted into a suitable vector and used. The type of a vector used in the present invention is not particularly limited. For example, it may be autonomously replicating vector (e.g., a plasmid, etc.), or it may be a vector which is incorporated into the genome in host cells when it is introduced into the host cells, and replicates with an incorporated chromosome.

The vector used in the present invention is preferably an expression vector. In an expression vector, elements necessary for transcription (e.g., a promoter, etc.) are functionally ligated to the gene of the present invention. A promoter is a DNA sequence having transcription activity in host cells, and it can appropriately be selected depending on the type of host cells.

Examples of a promoter capable of functioning in bacterial cells may include a Bacillus stearothermophilus maltogenic amylase gene promoter, a Bacillus licheniformis alpha-amylase gene promoter, a Bacillus amyloliquefaciens BAN amylase gene promoter, a Bacillus subtilis alkaline protease gene promoter, a Bacillus pumilus xylosidase gene promoter, a phage λ P_{R} or P_{L} promoter, and an Escherichia coli lac, trp, or lac promoter.

Examples of a promoter capable of functioning in mammalian cells may include an SV40 promoter, an MT-1 (metallothionein gene) promoter, and an adenovirus 2 major late promoter. Examples of a promoter capable of functioning in insect cells may include a polyhedrin promoter, a P10 promoter, an Autographa californica polyhedrosis basic protein promoter, a baculovirus immediate early gene 1 promoter, and a baculovirus 39K delayed-early gene promoter. Examples of a promoter capable of functioning in yeast host cells may include a promoter derived from a yeast glycolytic system gene, an alcohol dehydrogenase gene promoter, a TPI1 promoter, and an ADH2-4c promoter.

Examples of a promoter capable of functioning in filamentous cells may include an ADH3 promoter and a tpiA promoter.

The DNA of the present invention may be functionally ligated to a human growth hormone terminator, or in the case where a host is Mycomycete, the DNA may be functionally ligated to an appropriate terminator such as a TPI1 terminator or ADH3 terminator, as necessary. The recombinant vector of the present invention may also comprise elements such as a polyadenylation signal (e.g., those derived from SV40 or adenovirus 5E1b region), a transcription enhancer sequence (e.g., SV40 enhancer), and a translation enhancer sequence (e.g., those encoding adenovirus VA RNA).

The recombinant vector of the present invention may further comprise a DNA sequence enabling the vector to replicate in host cells. An example may include an SV40 replication origin (when the host cells are mammalian cells).

The recombinant vector of the present invention may further comprise a selective marker. Examples of a selective marker may include genes whose complements are deficient in host cells, such as dihydrofolate reductase (DHFR) or a Schizosaccharomyces pombe TPI gene, and drug resistant genes that are resistant to ampicillin, kanamycin, tetracycline, chloramphenicol, neomycin, hygromycin, etc.

A method of ligating the DNA of the present invention, a promoter, and a terminator and/or a secretory signal sequence, as desired, to one another, and inserting them into a suitable vector has been well known to those skilled in the art.

### (4) Transformant of the Present Invention, and Production of Protein using the Same

The DNA or recombinant vector of the present invention can be introduced into a suitable host, so as to prepare a transformant.

Any cells may be used as host cells into which the DNA or recombinant vector of the present invention is introduced, as long as they allow the DNA construct of the present invention to express therein. Examples of host cells may include bacteria, yeasts, Mycomycetes, and higher eukaryotes.

Examples of bacterial cells may include Gram-positive bacteria such as Bacillus or Streptomyces, and Gram-negative bacteria such as Escherichia coli. Transformation of these bacteria may be carried out by the protoplast method or known methods, using competent cells.

Examples of mammalian cells may include HEK293 cells, HeLa cells, COS cells, BHK cells, CHL cells, and CHO cells. A method of transforming mammalian cells and allowing a DNA sequence introduced into the cells to express therein has also been known. Examples of such a method may include the electroporation, the calcium phosphate method, and the lipofection method.

Examples of yeast cells may include cells belonging to Saccharomyces or Schizosaccharomyces. Examples of such cells may include Saccharomyces cerevisiae and Saccharomyces kluyveri. Examples of a method of introducing a recombinant vector into a yeast host may include the electroporation, the spheroplast method, and the lithium acetate method.

Examples of other fungal cells may include cells belonging to filamentous fungi such as Aspergillus, Neurospora, Fusarium, or Trichoderma. When filamentous fungi are used as host cells, transformation can be carried out by incorporating a DNA construct into a host chromosome to obtain recombinant host cells. Such a DNA construct can be incorporated into a host chromosome according to known methods such as homologous recombination or heterologous recombination.

When insect cells are used as host cells, a recombinant gene-introduced vector and baculovirus are co-introduced into insect cells, and recombinant virus is obtained in the culture supernatant of the insect cells. Thereafter, insect cells are infected with the recombinant virus, so that a protein is expressed (which is described in e.g. Baculovirus Expression Vectors, A Laboratory Manual; and Current Protocols in Molecular Biology, Bio/Technology, 6, 47 (1998)).

As an example of baculovirus, Autographa californica nuclear polyhedrosis virus infecting Mamestra insects can be used.

Examples of insect cells used herein may include Spodoptera frugiperda ovarian cells Sf 9 and Sf21 [Baculovirus Expression Vectors, A Laboratory Manual, W. H. Freeman and Company, New York (1992)], and Trichoplusia ni ovarian cells HiFive (manufactured by Invitrogen).

Examples of a method of co-introducing a recombinant gene-introduced vector and the above baculovirus into insect cells to prepare recombinant virus may include the calcium phosphate method and the lipofection method.

The above transformant is cultured in a nutrient medium under conditions enabling the expression of the introduced DNA construct. In order to isolate and purify the enzyme of the present invention from the culture of the transformant, common protein isolation and purification methods may be applied.

For example, where the enzyme of the present invention is expressed in a state where it is dissolved in cells, the cells are recovered by centrifugation after completion of the culture, and they are then suspended in a water-type buffer solution. Thereafter, the cells were disintegrated with an ultrasonic disintegrator or the like, so as to obtain a cell-free extract. A purified sample can be obtained from a supernatant obtained by centrifuging the above cell-free extract, using singly or in combination the following common protein isolation and purification methods: solvent extraction method, salting-out using ammonium sulfate or the like, desalting, precipitation method using organic solvents, anion exchange chromatography using resin such as diethylaminoethyl (DEAE) sepharose, cation exchange chromatography using resin such as S-Sepharose FF (manufactured by Pharmacia), hydrophobic chromatography using resin such as butyl sepharose or phenyl sepharose, gel filtration using a molecular sieve, affinity chromatography, chromatofocusing, electrophoresis such as isoelectric focusing, etc.

The present invention will be further specifically described in the following examples. However, these examples are not intended to limit the scope of the present invention.

### EXAMPLES

### Example 1

### O-glycan α2,8-sialyltransferase

The following reagents and samples were used in specific examples of the present invention. Fetuin, asialofetuin, bovine submaxillary mucin (BSM), α1-acid glycoprotein, ovomucoid, lactosyl ceramide (LacCer), GM3, GM1a, GD1a, GD1b, GT1b, CMP-NeuAc, 6′-sialyllactose, 3′-sialyl-N-acetyllactosamine, and Triton CF-54 were purchased from Sigma. 3′-sialyllactose and 6′-sialyl-N-acetyllactosamine were purchased from Calbiochem. N-acetylneuraminic acid (NeuAc), GM4, Gal, and N-acetylgalactosamine (GalNAc) were purchased from Wako Pure Chemical Industries, Ltd. GD3 was purchased from Snow Brand Milk Products Co., Ltd. GQ1b was purchased from Alexis Biochemicals. CMP-[¹⁴C]-NeuAc (12.0 GBq/mmol) was purchased from Amersham Pharmacia Biotech. Sialidases (NANase II, III) were purchased from Glyko Inc. N-glycanase (Glycopeptidase F) was purchased from Takara Shuzo Co., Ltd. [α-³²P]dCTP was purchased from NEN. Human Multiple tissue cDNA panel was purchased from Clontech. GM1b and its positional analogs, GSC-68, 2,3-sialylparagloboside (2,3-SPG), and 2,6-sialylparagloboside (2,6-SPG) were contributed from Prof. Makoto Kiso (Faculty of Agriculture, Gifu University). NeuAcα2,3Gal and NeuAcα2,6Gal were contributed from Dr. Hideki Ishida (The Noguchi Institute). An anti-GD3 monoclonal antibody KM641 was contributed from Dr. Kenya Shitara and Dr. Nobuo Hanai of Kyowa Hakko Kogyo Co., Ltd. In addition, an anti-NeuAcα2,8NeuAcα2,3Gal antibody S2-566 was purchased from Seikagaku Corp. Peroxidase-conjugated AffiniPure goat anti-mouse IgG+IgM (H+L) was purchased from Jackson Immuno Research. Desialylated (asialo) glycoproteins obtained by removing sialic acids from BSM, α1-acid glycoprotein, and ovomucoid were prepared by treating them at 80° C. for 1 hour in 0.02 N HCl.

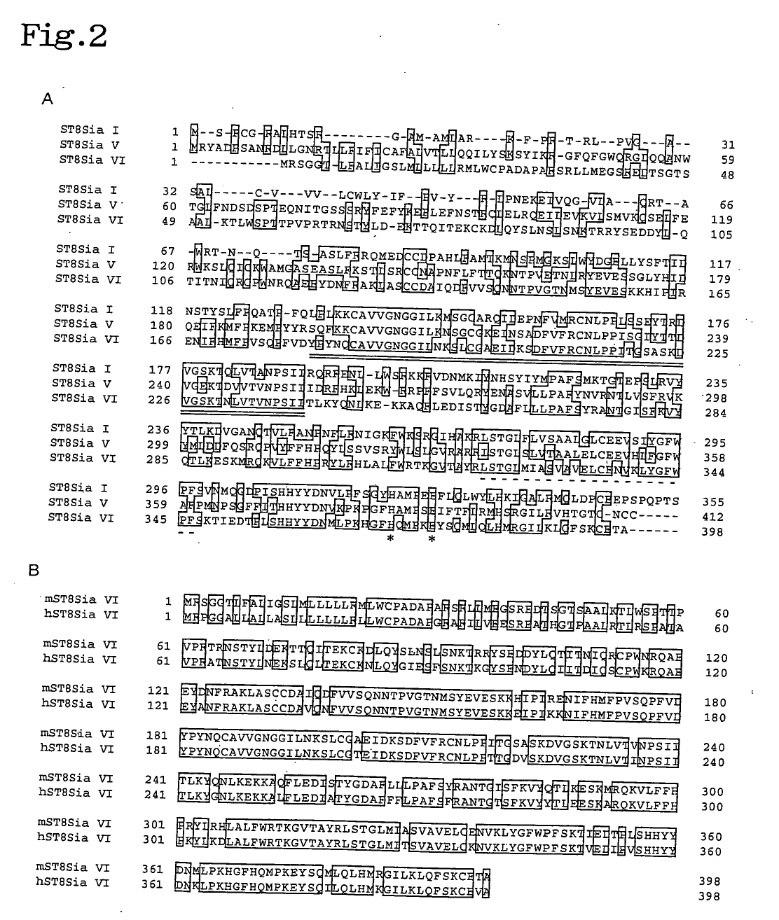

Using the amino acid sequence of mouse sialyltransferase ST8Sia V, a clone encoding a novel sialyltransferase showing a homology with the above enzyme has been searched against the database of expressed sequence tag (dbEST) of the National Center for Biotechnology Information. As a result, clones deposited under GenBank™ accession Nos. BE633149, BE686184, and BF730564 were obtained. Based on the information regarding the nucleotide sequences of these clones, two types of synthetic DNA fragments, 5′-CTTTTCTGGAGAACTAAAGG-3′ (corresponding to nucleotides 1001-1020 in FIG. 1A) (SEQ ID NO: 9) and 5′-AATTGCAGTTTGAGGATTCC-3′ (corresponding to a complementary strand of nucleotides 1232-1251 in FIG. 1A) (SEQ ID NO: 10) were prepared. Thereafter, in accordance with the method of Israel (Israel, D. I. (1993) Nucleic Acids Res. 21, 2627-2631), the cDNA library of each of mouse brain and heart was screened by the polymerase chain reaction method (PCR). As a result, a clone encoding a portion of a novel sialyltransferase was obtained from each cDNA library. In order to obtain a full-length clone, two types of synthetic DNA fragments 5′-TGGCTCAGGATGAGATCGGG-3′ (corresponding to nucleotides 68-87 in FIG. 1A) (SEQ ID NO: 11) and 5′-TACTAGCGCTCCCTGTGATTGG-3′ (corresponding to a complementary strand of nucleotides 725-746 in FIG. 1A) (SEQ ID NO: 12) were further prepared. Thereafter, using mouse kidney-derived cDNA as a template, DNA located between both the synthetic DNA fragments was amplified by PCR. The amplified fragment was ligated to a clone obtained from the mouse brain cDNA library, so as to obtain a full-length clone. This cDNA had a single open reading frame encoding type II transmembrane protein of 398 amino acids with an estimated molecular weight of 45,399. In addition, sialyl motifs conserved in sialyltransferases were present in the amino acid sequence thereof. This protein showed 42.0% and 38.3% homology with ST8Sia I and V, respectively, at an amino acid sequence level among known mouse sialyltransferases (FIG. 2A). As described below, since this protein had the activity of α2,8-sialyltransferase, it was named as the O-glycan α2,8-sialyltransferase of the present invention, ST8Sia VI.

On the other hand, in order to examine whether or not enzymes similar to the above enzyme are present in other mammals, using the sequence information of mouse ST8Sia VI, database was searched in the same manner as described above. As a result, it could be confirmed that similar enzymes are also present in human and rat. FIG. 1B shows the sequence information of human ST8Sia VI. Mouse ST8Sia VI showed a homology of 82.4% with human ST8Sia VI at an amino acid sequence level (FIG. 2B).

Subsequently, in order to examine enzymatic properties of ST8Sia VI, a secretory protein was produced. First, with regard to mouse ST8Sia VI, using two types of synthetic DNA fragments each containing a XhoI site, 5′-TGCTCTCGAGCCCAGCCGACGCGCCTGCCC-3′ (corresponding to nucleotides 141-170 in FIG. 1A) (SEQ ID NO: 13) and 5′-TATTCTCGAGCTAAGAAACGTTAAGCCGTT-3′ (corresponding to a complementary strand of nucleotides 1263-1293 in FIG. 1A) (SEQ ID NO: 14), a DNA fragment encoding the active domain of mouse ST8Sia VI was amplified by PCR with cloned full-length cDNA as a template. The amplified product was cleaved with XhoI, and a cleaved portion was inserted into the XhoI site of a mammalian expression vector, pcDSA. The obtained expression vector was named as pcDSA-mST8Sia VI.

On the other hand, with regard to human ST8Sia VI, first, using two types of synthetic DNA fragments, 5′-CAATTGACATATCTGAATGAGAAGTCGCTC-3′ (corresponding to nucleotides 293-315 in FIG. 1B) (SEQ ID NO: 15) and 5′-TACTAACATCTCCTGTGGTTGG-3′ (corresponding to a complementary strand of nucleotides 740-761 in FIG. 1B) (SEQ ID NO: 16), a DNA fragment was amplified by PCR with colon adenocarcinoma CX-1-derived cDNA from Human Tumor Multiple Tissue cDNA Panels (Clontech) as a template. Thereafter, using two types of synthetic DNA fragments, 5′-CCAGTGTCCCAGCCTTTTGT-3′ (corresponding to nucleotides 608-627 in FIG. 1B) (SEQ ID NO: 17) and 5′-TGAGTGGGGAAGCTTTGGTC-3′ (corresponding to a complementary strand of nucleotides 1407-1426 in FIG. 1B) (SEQ ID NO: 18), a DNA fragment was amplified by PCR in the same manner as described above. Thereafter, the two amplified DNA fragments were ligated to each other, using an EcoRI site that was common in both the amplified fragments, thereby obtaining a DNA fragment encoding the active domain of human ST8Sia VI. This fragment was inserted into the EcoRV site of a cloning vector pBluescript II SK(+), and thereafter, a fragment was cleaved with MunI and XhoI. The cleaved fragment was then inserted into the EcoRI-XhoI site of pcDSA. The obtained product was named as an expression vector pcDSA-hST8Sia VI.

pcDSA-mST8Sia VI and pcDSA-hST8Sia VI encode a secretory fusion protein comprising a signal peptide of mouse immunoglobulin IgM, Staphylococcus aureus protein A, and the active domain of mouse or human ST8Sia VI (which corresponds to amino acids 26-398 in the case of mouse ST8Sia VI and amino acids 68-398 in the case of human ST8Sia VI).

Using each expression vector and lipofectamine (Invitrogen), transient expression was carried out in COS-7 cells (Kojima, N. et al. (1995) FEBS Lett. 360, 1-4). The proteins of the present invention secreted from the cells into which each expression vector had been introduced were named as PA-mST8Sia VI (mouse) and PA-hST8Sia VI (human). PA-mST8Sia VI and PA-hST8Sia VI were adsorbed to IgG-Sepharose (Amersham Pharmacia Biotech), and were then recovered from medium. Sialyltransferase activity was measured as follows according to the method of Lee et al. (Lee, Y.-C. et al. (1999) J. Biol. Chem. 274, 11958-11967). A reaction solution (10 μl) containing 50 mM MES buffer (pH 6.0), 1 mM MgCl₂, 1 mM CaCl₂, 0.5% Triton CF-54, 100 μM CMP-[¹⁴C]-NeuAc, a glycoconjugate (which was added at 0.5 mg/ml in the case of glycolipids, and at 1 mg/ml in the case of glycoproteins or oligosaccharides), and a PA-mST8Sia VI or PA-hST8Sia VI suspension, was incubated at 37° C. for 3 to 20 hours. Thereafter, in the case of glycolipids, the reaction product was purified with a C-18 column (Sep-Pak Vac 100 mg; Waters) and the purified product was used as a sample, and in the case of oligosaccharides or glycoproteins, the reaction product was directly used as a sample. Thus, the obtained samples were subjected to analysis. In the case of oligosaccharides or glycolipids, the sample was spotted on a silica gel 60 HPTLC plate (Merck), and was then developed with a developing solvent consisting of ethanol:pyridine:n-butanol:water:acetic acid=100:10:10:30:3 (for oligosaccharides), a developing solvent consisting of 1-propanol:ammonia water:water=6:1:2.5 (for oligosaccharides), or a developing solvent consisting of chloroform:methanol: 0.02% CaCl₂=55:45:10 (for glycolipids). In the case of glycoproteins, analysis was carried out by SDS-polyacrylamide gel electrophoresis. The obtained radioactivities were visualized with a BAS2000 radio image analyzer (Fuji Film) and then quantified.

Table 1 shows substrate specificity of PA-mST8Sia VI and PA-hST8Sia VI.

TABLE I
Acceptor substrate specificity of ST8Sia VI Using PA-mST8Sia VI and PA-hST8Sia VI, specificity against various
acceptor substrates was examined. The concentration of the substrates is 0.5 mg/ml in the case of glycolipids,
and 1 mg/ml in the case of glycoproteins, monosaccharides and oligosaccharides. The relative activity was calculated
by taking incorporation obtained with Fetuin (PA-mST8Sia VI is 2.06 pmol/h/(ml enzyme solution), and PA-hST8Sia VI
is 0.204 pmol/h/(ml enzyme solution)) as 100. R represents the remainder of the N-linked sugar chain. ND: not determined

| | | Relative rate (%) | |
|---|---|---|---|
| | | | Human |
| | | Mouse | ST8Sia |
| Acceptor | Representative structures of carbohydrates | ST8SiaVI | VI |
| Glycoproteins | | 100 | 100 |
| Fetuin | | | |
| | NeuAcα2,3Galβ1,3GalNAc-O-Ser/Thr | | |
| | NeuAcα2,3Galβ1,3(NeuAcα2,6)GalNAc-O-Ser/Thr | | |
| | NeuAcα2,6(3)Galβl,4GlcNAc-R | | |
| Asialofetuln | | 0 | 0 |
| α1-Acid glycoprotein | NeuAcα2,6(3)Galβ1,4GlcNAc-R | 0 | 0 |
| Asialo- α1-Acid glycoprotein | | 0 | 0 |
| BSM | NeuAcα2,6GalNAc-O-Ser/Thr | 375 | 24.2 |
| | GlcNAcβ1,3(NeuAcα2,6)GalNAc-O-Ser/Thr | | |
| Asialo-BSM | | 0 | 0 |
| Ovomucoid | NeuAcα2,3Galβ1,4GlcNAc-R | 6.2 | 12.3 |
| Asialoovomucoid | | 0 | 0 |
| Glycollpids | | | |
| Lactosylceramide | Galβ1,4Glcβ1-Cer | 0 | ND |
| GM4 | NeuAcα2,3Galβ1-Cer | 1.0 | ND |
| GM3 | NeuAcα2,3Galβ1,4Glcβ1-Cer | 13.0 | 1.6 |
| GM1a | Galβ1,3GalNAcβ1,4(NeuAcα2,3)Galβ1,4Glcβ1-Cer | 0 | ND |
| GD1a | NeuAcα2,3Galβ1,3GalNAcβ1,4(NeuAca2,3)Galβ1,4Glcβ1-Cer | 6.0 | 1.8 |
| GD3 | NeuAcα2,8NeuAcα2,3Galβ1,4Glcβ1-Cer | 0 | 0 |
| GD1b | Galβ1,3GalNAcβ1,4(NeuAcα2,8NeuAcα2,3)Galβ1,4Glcβ1-Cer | 0 | ND |
| GT1b | NeuAcα2,8Galβ1,3GalNAcβ1,4(NeuAcα2,8NeuAcα2,3)Galβ1,4Glcβ1-Cer | 1.1 | 2.2 |
| GQ1b | NeuAcα2,8NeuAcα2,8Galβ1,3GalNAcβ1,4(NeuAcα2,8NeuAcα2,3)Galβ1,4Glcβ1-Cer | 0 | 0 |
| GM1b | NeuAcα2,3Galβ1,3GalNAcβ1,4Galβ1,4Glcβ1-Cer | 1.0 | ND |
| GSC-68 | NeuAcα2,6Galβ1,3GalNAcβ1,4Galβ1,4Glcβ1-Cer | 2.6 | ND |
| 2,3-SPG | NeuAcα2,3Galβ1,4GlcNAcβ1,3Galβ1,4Glcβ1-Cer | 3.5 | ND |
| 2,6-SPG | NeuAcα2,6Galβ1,4GlcNAcβ1,3Galβ1,4Glcβ1-Cer | 0.98 | ND |
| Monosaccharides and oligosaccharides | | | |
| 3′-Sialyllactose | NeuAcα2,3Galβ1,4Glc | 629 | 69.9 |
| 6′-Sialyllactose | NeuAcα2,6Galβ1,4Glc | 91.5 | 10.7 |
| 3′-Sialyl-N-acetyllactosamine | NeuAcα2,3Galβ1,4GlcNAc | 411 | ND |
| 6′-Sialyl-N-acetyllactosamine | NeuAcα2,6Galβ1,4GlcNAc | 88.7 | ND |
| 3′-Sialylgalactose | NeuAcα2,3Gal | 13.9 | ND |
| 6′-Sialylgalactose | NeuAcα2,6Gal | 2.0 | ND |
| N-Acetylneuraminic acid | NeuAc | 0 | ND |
| Galactose | Gal | 0 | ND |
| N-Acetylgalactosamine | GalNAc | 0 | ND |

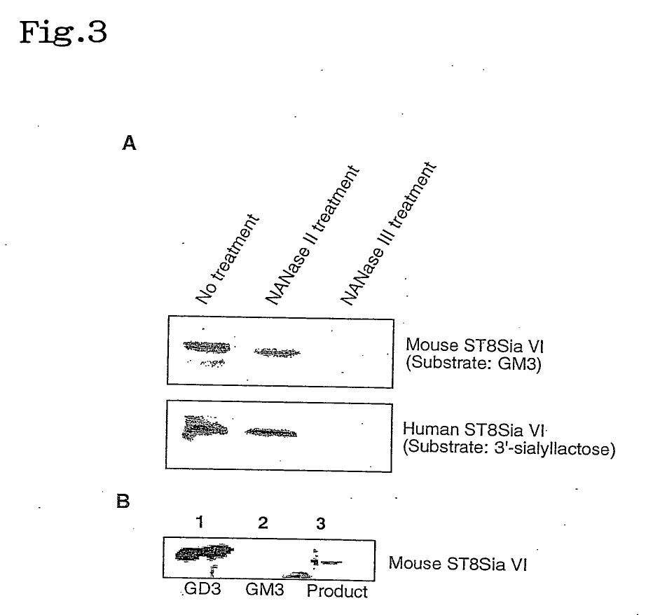

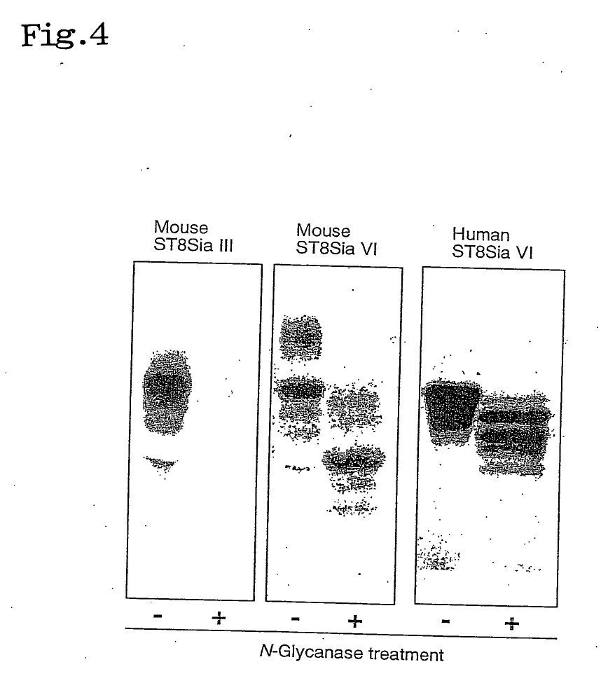

PA-mST8Sia VI showed activity on glycolipids having a structure “NeuAcα2,3(6)Gal-” at the nonreducing end thereof, such as GM4, GM3, GD1a, GT1b, GM1b, GSC-68, 2,3-SPG, or 2,6-SPG. When GM3 was used as a substrate, the incorporated sialic acid of the reaction product was not cleaved with sialidase (NANase II), which specifically cleaves α2,3- and α2,6-linked sialic acid. However, the incorporated sialic acid was cleaved with sialidase (NANase III), which specifically cleaves α2,3-, α2,6-, α2,8- and α2,9-linked sialic acids (FIG. 3A). In addition, it was confirmed by TLC immunostaining using an anti-GD3 monoclonal antibody KM641 (Saito, M. et al. (2000) Biochim. Biophys. Acta 1523, 230-235) that this reaction product was GD3 into which sialic acid had been introduced through an α2,8-linkage (FIG. 3B). Thus, it was clarified that PA-mST8Sia VI transfers sialic acid through an α2,8-linkage.

On the other hand, where a glycoprotein was used as a substrate (Table 1), PA-mST8Sia showed the highest activity toward BSM, which contains only O-glycans as glycoconjugate. PA-mST8Sia also showed activity toward Fetuin, which contains both O-glycans and N-glycans and toward Ovomucoid, which contains only N-glycans. However, the activity toward Ovomucoid was lower than that toward a protein containing O-glycans. Moreover, PA-mST8Sia VI showed no activity on asialoglycoproteins. Furthermore, from an experiment wherein monosaccharide or oligosaccharide was used as a substrate (Table 1), it was found that the minimum sugar chain unit, which was recognized by PA-mST8Sia VI as a substrate, is NeuAcα2,3(6)Gal.

It was found by an N-glycanase treatment that when Fetuin was used as a substrate, the majority of sialic acid, which was newly introduced by PA-mST8Sia VI, was incorporated into O-glycans (FIG. 4). That is, when Fetuin was sialylated by PA-mST8Sia VI with [¹⁴C]-NeuAc, and the sialylated product was then treated with N-glycanase, which releases N-glycans from a peptide portion. The majority (82.7%) of radioactivity was still kept in the Fetuin after this treatment. This fact shows that the majority of sialic acid introduced by PA-mST8Sia VI was incorporated into O-glycans. On the other hand, the same experiment was carried out using mouse ST8Sia III, which used N-glycans of Fetuin as substrates. As a result, it was found that radioactivity completely disappeared.

Moreover, in order to clarify the substrate specificity and substrate selectivity of PA-mST8Sia VI, the Km and Vmax values for BSM and GM3, respectively, were obtained. With regard to BSM, the Km value was 0.03 mM, the Vmax value was 23.8 pmol/h/ml enzyme solution, and the Vmax/Km value was 793. With regard to GM3, the Km value was 0.5 mM, the Vmax value was 0.67 pmol/h/ml enzyme solution, and the Vmax/Km value was 1.34. These results show that, for PA-mST8Sia VI, O-glycans are much more preferable substrates than glycolipids or N-glycans.

PA-hST8Sia VI has the same enzymatic properties as those described above, although differences are somewhat found in activity values (Table 1, and FIGS. 3A and 4). Accordingly, it can be said that ST8Sia VI derived from various types of animals had substrate specificity different from that of the conventional α2,8-sialyltransferases.

In addition, concerning mouse ST8Sia VI, the in vivo enzymatic activity of the full-length clone was also examined (FIG. 5). A 1.4-kb NotI-ApaI fragment containing a region encoding the full-length mouse ST8Sia VI was inserted into the NotI-ApaI site of an expression vector pRc/CMV, and it was named as pRc/CMV-ST8Sia VI. The vector pRc/CMV-ST8Sia VI was introduced into COS-7 cells using lipofectamine. Ganglioside was extracted from the cells, and it was then subjected to TLC immunostaining, using a monoclonal antibody S2-566 which recognizes an NeuAcα2,8NeuAcα2,3Gal structure (FIG. 5A). As a result, it was found that the amount of ganglioside having an NeuAcα2,8NeuAcα2,3Gal structure was significantly increased in the cells into which pRc/CMV-ST8Sia VI had been introduced. Moreover, with regard to glycoproteins in the cells, NeuAcα2,8NeuAcα2,3Gal structures were also newly formed on O-glycans of the cells into which pRc/CMV-ST8Sia VI had been introduced (FIG. 5B). These results show that mouse ST8Sia VI functions as α2,8-sialyltransferase in vivo.

Mouse ST8Sia VI is expressed mainly in the kidney, heart, spleen, or the like (FIG. 6A), but human ST8Sia VI is expressed mainly in the placenta, various types of embryonic tissues, various types of tumor cells, or the like (FIG. 6B).

### Example 2

### β-galactoside α2,6-sialyltransferase

The following reagents and samples were used in specific examples of the present invention. Fetuin, asialofetuin, bovine submaxillary mucin (BSM), α1-acid glycoprotein, ovomucoid, lactosyl ceramide (LacCer), GA1, GM3, GM1a, Galβ1,3GalNAc, Galβ1,3GlcNAc, Galβ1,4GlcNAc, Triton CF-54, and β-galactosidase (derived from bovine testis) were purchased from Sigma. Paragloboside and lactose were purchased from Wako Pure Chemical Industries, Ltd. CMP-[¹⁴C]-NeuAc (12.0 GBq/mmol) was purchased from Amersham Pharmacia Biotech. Lacto-N-tetraose, Lacto-N-neotetraose, and sialidases (NANase I, II) were purchased from Glyko Inc. [α-³²P]dCTP was purchased from NEN. Human and mouse Multiple tissue cDNA panels were purchased from Clontech. Desialylated (asialo) glycoproteins obtained by removing sialic acids from BSM, α1-acid glycoprotein, and ovomucoid were prepared by treating them at 80° C. for 1 hour in 0.02 N HCl.

Using the amino acid sequence of human sialyltransferase ST6Gal I, a clone encoding a novel sialyltransferase showing a homology with the above enzyme has been searched against the database of expressed sequence tag (dbEST) of the National Center for Biotechnology Information. As a result, EST clones deposited under GenBank™ accession Nos. BE613250, BE612797, and BF03852 were obtained. These clones were purchased from the I. M. A. G. E. Consortium. Using the information of these nucleotide sequences, the dbEST database and the high throughput genomic sequence database of the human genome were searched, and the related EST clones and the genomic nucleotide sequence information of this gene were obtained (Accession Nos. H94068, AA514734, BF839115, AA210926, AA385852, H94143, and BF351512 (EST clones), and AC016994 (genome sequence)). Based on the information on the above nucleotide sequences, primers used for the polymerase chain reaction method (PCR) were synthesized. Using these primers, PCR was performed with human colon-derived cDNA as a template. Thereafter, the amplified fragment was ligated to the DNA fragment derived from the obtained EST clone, so as to obtain a clone containing the full-length coding region (FIG. 7A). This cDNA had a single open reading frame which encodes a type-II transmembrane protein of 529 amino acids and it has an estimated molecular weight of 60,157. It was predicted from the hydropathy plot that a transmembrane domain exists in the region corresponding to amino acids 12-30 (FIG. 7B). The sialyl motifs conserved in sialyltransferases were present in the amino acid sequence of the present protein. Moreover, among the known human sialyltransferases, the present protein showed the highest homology (48.9%) with ST6Gal I at an amino acid sequence level (FIG. 9A), but it showed only approximately 21% to 36% homology with sialyltransferases belonging to other families. As described below, since this protein had the activity of β-galactoside α2,6-sialyltransferase, it was named as the β-galactoside α2,6-sialyltransferase of the present invention, ST6Gal II. In addition, there was a short-form clone of human ST6Gal II, having a different sequence from the middle of sialyl motif S, which was considered to be a splicing variant (FIG. 7A).

On the other hand, in order to examine whether or not enzymes similar to the above enzyme are present also in other mammals, database was searched in the same manner as described above using the sequence information of human ST6Gal II. As a result, it could be confirmed that similar enzymes are also present in mice. Thus, cloning was also carried out on mice. Using two types of synthetic DNA fragments, 5′-GACAATGGGGATGAGTTTTTTACATCCCAG-3′ (corresponding to nucleotides 321-350 in FIG. 8A) (SEQ ID NO: 19) and 5′-CGATTTCCTCCCCCAAGGAGGAGTTCAGG-3′ (corresponding to a complementary strand of nucleotides 864-893 in FIG. 8A) (SEQ ID NO: 20), a DNA fragment was amplified by PCR with mouse 14-day-old embryo-derived cDNA as a template. Moreover, using two types of synthetic DNA fragments, 5′-ACGTTGGACGGCAGAGAGGCGCCCTTCTCG-3′ (corresponding to nucleotides 774-803 in FIG. 8A) (SEQ ID NO: 21) and 5′-ACCTTATTGCACATCAGTTCCCAAGAGTTC-3′ (corresponding to a complementary strand of nucleotides 1582-1611 in FIG. 8A) (SEQ ID NO: 22), a DNA fragment was amplified by PCR in the same manner as described above. Thereafter, the two amplified DNA fragments were ligated to each other, using a KpnI site that was common in both the amplified fragments. Thereafter, another DNA fragment which was amplified by PCR using two types of synthetic DNA fragments 5′-CAATGAAACCACACTTGAAGCAATGGCGAC-3′ (corresponding to nucleotides 1-30 in FIG. 8A) (SEQ ID NO: 23) and 5′-CGCAACAAAAAAATAGCTATCTTCCTCGGG-3′ (corresponding to a complementary strand of nucleotides 381-410 in FIG. 8A) (SEQ ID NO: 24), was further ligated to the above ligated fragment, using an Aor51HI site common in both the DNA fragments, so as to obtain a DNA fragment encoding the full-length mouse ST6Gal II. The obtained DNA fragment was then inserted into a cloning vector pBluescript II SK(+). FIG. 8A shows the sequence information of mouse ST6Gal II. Mouse ST6Gal II consisted of 524 amino acids, and a portion corresponding to a stem region in mouse ST6Gal II was approximately 5 amino acids shorter than that in human ST6Gal II. It was predicted from the hydropathy plot that the transmembrane domain of the present protein exists in a region corresponding to amino acids 12-30 (FIG. 8B). Human ST6Gal II showed 77.1% homology with mouse ST6Gal II at an amino acid sequence level (FIG. 9B).

Subsequently, in order to examine enzymatic properties of ST6Gal II, a secretory protein was produced. First, with regard to human ST6Gal II, a XhoI site was introduced immediately downstream of the DNA portion encoding the transmembrane domain using a synthetic DNA fragment containing a XhoI site, 5′-TCATCTACTTCACCTCGAGCAACCCCGCTG-3′ (corresponding to nucleotides 255-284 in FIG. 7A) (SEQ ID NO: 25). Using this site and a XhoI site of the pBluescript II SK(+), the XhoI fragment encoding the stem region and active domain of ST6Gal II was prepared. This XhoI fragment was then inserted into the XhoI site of a mammalian expression vector pcDSA. The obtained expression vector was named as pcDSA-hST6Gal II. On the other hand, with regard to mouse ST6Gal II, using a synthetic DNA fragment containing a MunI site, 5′-CATCCAATTGACCAACAGCAATCCTGCGGC-3′ (corresponding to nucleotides 83-112 in FIG. 8A) (SEQ ID NO: 26) instead of the synthetic DNA fragment used in the above cloning, 5′-CAATGAAACCACACTTGAAGCAATGGCGAC-3′ (corresponding to nucleotides 1-30 in FIG. 8A) (SEQ ID NO: 23), the MunI-XhoI fragment encoding the stem region and active domain of mouse ST6Gal II was prepared. This fragment was then inserted into the EcoRI-XhoI site of pcDSA. The thus obtained vector was named as an expression vector pcDSA-mST6Gal II.

pcDSA-mST6Gal II and pcDSA-hST6Gal II encode a secretory fusion protein comprising a signal peptide of mouse immunoglobulin IgM, Staphylococcus aureus protein A, and an active domain of mouse or human ST6Gal II (which corresponds to amino acids 33-529 in the case of human ST6Gal II, and amino acids 31-524 in the case of mouse ST6Gal II).

Using each expression vector and lipofectamine (Invitrogen), transient expression was carried out in COS-7 cells (Kojima, N. et al. (1995) FEBS Lett. 360, 1-4). The proteins of the present invention secreted from the cells into which each expression vector had been introduced were named as PA-hST6Gal II (human) and PA-mST6Gal II (mouse). PA-hST6Gal II and PA-mST6Gal II were adsorbed to IgG-Sepharose (Amersham Pharmacia Biotech), and were then recovered from medium. Sialyltransferase activity was measured as follows according to the method of Lee et al. (Lee, Y.-C. et al. (1999) J Biol. Chem. 274, 11958-11967). A reaction mixture (10 μl) containing 50 mM MES buffer (pH 6.0), 1 mM MgCl₂, 1 mM CaCl₂, 0.5% Triton CF-54, 100 μM CMP-[¹⁴C]-NeuAc, a substrate sugar chain (which was added at 0.5 mg/ml in the case of glycolipids, and at 1 mg/ml in the case of glycoproteins or oligosaccharides), and a PA-hST6Gal II or PA-mST6Gal II suspension, was incubated at 37° C. for 3 to 20 hours. Thereafter, in the case of glycolipids, the reaction product was purified with a C-18 column (Sep-Pak Vac 100 mg; Waters) and the purified product was used as a sample. In the case of oligosaccharides or glycoproteins, the reaction product was directly used as a sample. Thus, the obtained sample was subjected to analysis. In the case of oligosaccharides or glycolipids, the sample was spotted on a silica gel 60 HPTLC plate (Merck), and it was then developed with a developing solvent consisting of 1-propanol:ammonia water:water=6:1:2.5 (for oligosaccharides), or a developing solvent consisting of chloroform:methanol: 0.02% CaCl₂=55:45:10 (for glycolipids). In the case of glycoproteins, analysis was carried out by SDS-polyacrylamide gel electrophoresis. The obtained radioactivities were visualized with a BAS2000 radio image analyzer (Fuji Film) and then quantified.

Table 2 shows substrate specificity of PA-hST6Gal II and PA-mST6Gal II.

TABLE 2
Substrate specificity of ST6Gal II Using PA-hST6Gal II and PA-mST6Gal II, specificity
against various substrates was examined. The concentration of the substrates is 0.5 mg/ml
in the case of glycolipids, and 1 mg/ml in the case of glycoproteins, monosaccharides and
oligosaccharides. The relative activity was calculated by taking the incorporation obtained
with Gal β 1,4GlcNAc as 100. R represents the remainder of the N-linked sugar chain.

| | | Relative rate (%) | | |
|---|---|---|---|---|
| | | Mouse | Human | Human |
| Acceptors | Representative structures of carbohydrates | ST6Gal II | ST6Gal II | ST6Gal I |
| Oligosaccharides | | | | |
| Type II | Galβ1,4GlcNAc | 100* | 1O0** | 100*** |
| Type I | Galβ1,3GlcNAc | 0 | 0 | 4.2 |
| Type III | Galβ1,3GalNAc | 0 | 0 | 0 |
| Lactose | Galβ1,4Glc | 0 | 0 | 8.7 |
| Lacto-N-tetraose | Galβ1,3GlcNAcβ1,3Galβ1,4Glc | 0 | 0 | 31.1 |
| Lacto-N-neotetraose | Galβ1,4GlcNAcβ1,3Galβ1,4Glc | 128.8 | 86.2 | 101.6 |
| Glycoproteins | | | | |
| Fetuin | NeuAcα2,3Galβ1,3GalNAc-O-Ser/Thr | 0 | 0 | 13.0 |
| | NeuAcα2,3Galβ1,3(NeuAcα2,6)GaINAc-O-Ser/Thr | | | |
| | NeuAcα2,6(3)Galβ1,4GlcNAc-R | | | |
| Asialofetuin | 21.0 | 3.9 | 95.0 | |
| BSM | NeuAcα2,6GalNAc-O-Ser/Thr | 0 | 0 | 0 |
| | GlcNAcβ1,3(NeuAcα2,6)GalNAc-O-Ser/Thr | | | |
| Asialo-BSM | 0 | 0 | 0 | |
| Ovomucoid | NeuAcα2,3Galβ1,4GlcNAc-R | 0 | 0 | 9.0 |
| Asialoovomucoid | 0 | 0 | 12.7 | |
| αl-Acid glycoprotein | NeuAcα2,6(3)Galβ1,4GlcNAc-R | 0.75 | 1.2 | 37.1 |
| Asialo- αl-Acid glycoprotein | 12.3 | 1.2 | 93.0 | |
| Glycolipids | | | | |
| Lactosylceramide | Galβ1,4Glcβ1-Cer | 0 | 0 | 0 |
| GA1 | Galβ1,3GalNAcβ1,4Galβ1,4Glcβ1-Cer | 0 | 0 | 0 |
| GM1a | Galβ,3GalNcβ1,4(NeuAca2,3)Galβ1,4Glcβ1-Cer | 0 | 0 | 0 |
| GM3 | NeuAcα2,3Galβ1,4Glcβ1-Cer | 0 | 0 | 0 |
| Paragloboside | Galβ1,4GlcNAcβ1,3Galβ1,4Glcβ1-Cer | 0 | 0 | 0.3 |

*, 2.74 pmol/h/ml medium.
**, 1.03 pmol/h/ml medium.
***, 8.14 pmol/h/ml medium.
NeuAc,N-acetylneuraminic acid.
Cer, ceramide.

Both the enzymes showed activity only on oligosaccharides having a Galβ1,4GlcNAc structure at the nonreducing end thereof (FIG. 10). Moreover, the enzymes also showed weak activity on glycoproteins, which were likely to have the above structure. In contrast, there were no glycolipids, which could be substrates of both the enzymes, as far as the inventors have examined. The activity of human ST6Gal I on oligosaccharides was also examined for comparison. As a result, human ST6Gal I showed activity not only on oligosaccharides having a Galβ1,4GlcNAc structure, but also on lactose, Lacto-N-tetraose, etc. (FIG. 10). Moreover, ST6Gal I showed activity on a wide range of glycoproteins and glycolipids (Table 2). These results show that ST6Gal II has higher selectivity than ST6Gal I in terms of substrate specificity. Furthermore, it was confirmed that a short-form protein, which is a splicing variant of human ST6Gal II, had no enzyme activity (FIG. 10).

When sialic acid is transferred into Galβ1,4GlcNAc by PA-hST6Gal II or PA-mST6Gal II, as in the case of ST6Gal I, the incorporated sialic acid of the reaction product was not cleaved with sialidase (NANase I), which specifically cleaves α2,3-linked sialic acids. However, the incorporated sialic acid was cleaved with sialidase (NANase II), which specifically cleaves α2,3- and α2,6-linked sialic acids (FIG. 11A). Moreover, this reaction product showed the same mobility as that of 6′-sialyl-N-acetyllactosamine in TLC, and even after the reaction product was treated with galactosidase, there were no changes in its mobility in TLC (FIG. 11B). Accordingly, it was considered that the reaction product was 6′-sialyl-N-acetyllactosamine obtained by introducing sialic acid into galactose through an α2,6-linkage. As stated above, it was found that ST6Gal II transfers sialic acid into galactose through an α2,6-linkage. It was considered that its particularly preferred substrate is an oligosaccharide having a Galβ1,4GlcNAc structure at the nonreducing end thereof.

Further, the expression patterns of human ST6Gal I and ST6Gal II in various tissues were examined by PCR, using ST6Gal I-specific primers (5′-TTATGATTCACACCAACCTGAAG-3′ (SEQ ID NO: 27) and 5′-CTTTGTACTTGTTCATGCTTAGG-3′ (SEQ ID NO: 28); the size of a PCR amplified fragment: 372 bp), and ST6Gal II-specific primers (5′-AGACGTCATTTTGGTGGCCTGGG-3′ (corresponding to nucleotides 1264-1286 in FIG. 7A) (SEQ ID NO: 29) and 5′-TTAAGAGTGTGGAATGACTGG-3′ (corresponding to nucleotides 1745-1765 in FIG. 7A) (SEQ ID NO: 30); the size of a PCR amplified fragment: 502 bp) (FIG. 12A). As a result, it was found that human ST6Gal I was expressed in almost all tissues, but that human ST6Gal II was expressed at an extremely low level or was not expressed at all in tissues other than the small intestine, large intestine, or fetal brain. Moreover, it was also found that human ST6Gal I was expressed in various types of tumor cells, but that the expression of ST6Gal II was not detected in tumor cells (FIG. 12B). The expression pattern of mouse ST6Gal II was examined in the same above manner, using mouse ST6Gal II-specific primers (5′-CAATGAAACCACACTTGAAGCAATGGCGAC-3′ (corresponding to nucleotides 1-30 in FIG. 8A) (SEQ ID NO: 23) and 5′-CGCAACAAAAAAATAGCTATCTTCCTCGGG-3′ (corresponding to a complementary strand of nucleotides 381-410 in FIG. 8A) (SEQ ID NO: 24); the size of a PCR amplified fragment: 410 bp). As a result, it was found that the expression of mouse ST6Gal II was observed in the brain and embryo, but that the expression thereof was observed at an extremely low level or was not observed at all in other tissues (FIG. 12C). These results suggest that ST6Gal I and ST6Gal II play different roles in vivo.

### INDUSTRIAL APPLICABILITY

The present invention provides a novel enzyme O-glycan α2,8-sialyltransferase, and a novel protein having an active portion of the enzyme and being extracellularly secreted. The enzyme and protein of the present invention have the activity of O-glycan α2,8-sialyltransferase. Accordingly, it is useful as a reagent for introducing a human-type sugar chain into a protein, for example. In addition, the O-glycan α2,8-sialyltransferase of the present invention is useful also as a medicament for treating hereditary diseases caused by deficiency of sugar chains specific for humans. Moreover, the O-glycan α2,8-sialyltransferase of the present invention can also be used as a medicament which acts for suppression of cancer metastasis, prevention of virus infection, suppression of inflammatory response, or activation of neural cells. Furthermore, the O-glycan α2,8-sialyltransferase of the present invention is useful also as a reagent used in studies for increasing physiological action by adding sialic acid to drugs or the like.

Still further, the present invention provides a novel enzyme β-galactoside α2,6-sialyltransferase and a novel protein having an active portion of the enzyme and being extracellularly secreted. The enzyme and protein of the present invention has the activity of β-galactoside α2,6-sialyltransferase, and it thereby becomes possible to selectively introduce sialic acid through an α2,6-linkage into galactose such as oligosaccharide having a Galβ1,4GlcNAc structure. The β-galactoside α2,6-sialyltransferase ST6Gal II of the present invention is useful as a therapeutic agent for treating hereditary diseases caused by deficiency of specific sugar chains synthesized by the present enzyme, as an agent acting for suppression of cancer metastasis, prevention of virus infection, suppression of inflammatory response, or activation of neural cells, or as a reagent used in studies for increasing physiological action or inhibiting hydrolytic activity of glycolytic enzymes by adding sialic acid to sugar chains.

1. O-glycan α2,8-sialyltransferase having substrate specificity and substrate selectivity,
wherein the enzyme has substrate specificity wherein the substrates of the enzyme are glycoconjugates having a Siaα2,3(6)Gal structure wherein Sia represents sialic acid and Gal represents galactose at the terminus thereof; and
wherein the enzyme has substrate selectivity wherein the enzyme incorporates sialic acids into O-glycans more preferentially than into glycolipids or N-glycans.

2. O-glycan α2,8-sialyltransferase having either one of the following amino acid sequences:
(1) an amino acid sequence shown in SEQ ID NO: 1 or 3; or
(2) an amino acid sequence comprising a deletion, substitution, and/or addition of one or several amino acids with respect to the amino acid sequence shown in SEQ ID NO: 1 or 3, and having O-glycan α2,8-sialyltransferase activity.

3. O-glycan α2,8-sialyltransferase gene encoding the amino acid sequence of the O-glycan α2,8-sialyltransferase according to claim 2.

4. The O-glycan α2,8-sialyltransferase gene according to claim 3 which has any one of the following nucleotide sequences:
(1) a nucleotide sequence corresponding to a portion between nucleotide 77 and nucleotide 1270 of a nucleotide sequence shown in SEQ ID NO: 2;
(2) a nucleotide sequence comprising a deletion, substitution, and/or addition of one or several nucleotides with respect to the nucleotide sequence corresponding to a portion between nucleotide 77 and nucleotide 1270 of the nucleotide sequence shown in SEQ ID NO: 2, and encoding a protein having O-glycan α2,8-sialyltransferase activity;
(3) a nucleotide sequence corresponding to a portion between nucleotide 92 and nucleotide 1285 of a nucleotide sequence shown in SEQ ID NO: 4; and
(4) a nucleotide sequence comprising a deletion, substitution, and/or addition of one or several nucleotides with respect to the nucleotide sequence corresponding to a portion between nucleotide 92 and nucleotide 1285 of the nucleotide sequence shown in SEQ ID NO: 4, and encoding a protein having O-glycan α2,8-sialyltransferase activity.

5. A recombinant vector comprising the O-glycan α2,8-sialyltransferase gene according to claim 3.

6. The recombinant vector according to claim 5 which is an expression vector.

7. A transformant transformed with the recombinant vector according to claim 5.

8. A method for producing O-glycan α2,8-sialyltransferase wherein the transformant of claim 7 is cultured and O-glycan α2,8-sialyltransferase is collected from the culture.

9. A protein which comprises an active domain of O-glycan α2,8-sialyltransferase having any one of the following amino acid sequences:
(1) an amino acid sequence corresponding to a portion between positions 26 and 398 of the amino acid sequence shown in SEQ ID NO: 1;
(2) an amino acid sequence comprising a deletion, substitution, and/or addition of one or several amino acids with respect to the amino acid sequence corresponding to a portion between positions 26 and 398 of the amino acid sequence shown in SEQ ID NO: 1, and having O-glycan α2,8-sialyltransferase activity;
(3) an amino acid sequence corresponding to a portion between positions 68 and 398 of the amino acid sequence shown in SEQ ID NO: 3; and
(4) an amino acid sequence comprising a deletion, substitution, and/or addition of one or several amino acids with respect to the amino acid sequence corresponding to a portion between positions 68 and 398 of the amino acid sequence shown in SEQ ID NO: 3, and having O-glycan α2,8-sialyltransferase activity.

10. An extracellular secretory protein, comprising a polypeptide portion which is an active domain of the O-glycan α2,8-sialyltransferase of claim 1, and a signal peptide, and has O-glycan α2,8-sialyltransferase activity.

11. A gene encoding the protein according to claim 9.

12. A recombinant vector comprising the gene according to claim 11.

13. The recombinant vector according to claim 12 which is an expression vector.

14. A transformant transformed with the recombinant vector according to claim 12.

15. A method for producing a protein comprising an active domain of O-glycan α2,8-sialyltransferase wherein the transformant of claim 14 is cultured and the protein is collected from the culture.

16. β-galactoside α2,6-sialyltransferase having activity and substrate specificity,
wherein the activity comprises enzyme transfer of sialic acid through an α2,6 linkage into the galactose portion of a sugar chain having a galactose β1,4N-acetylglucosamine structure at the terminus thereof; and
wherein the enzyme has substrate specificity wherein the substrate of the enzyme is a sugar chain having a galactose β1,4N-acetylglucosamine structure at the terminus thereof, and lactose and a sugar chain having a galactose β1,3N-acetylglucosamine structure at the terminus thereof are not the substrate of the enzyme.

17. β-galactoside α2,6-sialyltransferase having either one of the following amino acids:
(1) an amino acid sequence shown in SEQ ID NO: 5 or 7; or
(2) an amino acid sequence comprising a deletion, substitution, and/or addition of one or several amino acids with respect to the amino acid sequence shown in SEQ ID NO: 5 or 7, and having β-galactoside α2,6-sialyltransferase activity.

18. A β-galactoside α2,6-sialyltransferase gene encoding the amino acid sequence of the β-galactoside α2,6-sialyltransferase according to claim 17.

19. The β-galactoside α2,6-sialyltransferase gene according to claim 18 which has any one of the following nucleotide sequences:
(1) a nucleotide sequence corresponding to a portion between nucleotide 176 and nucleotide 1762 of a nucleotide sequence shown in SEQ ID NO: 6;
(2) a nucleotide sequence comprising a deletion, substitution, and/or addition of one or several nucleotides with respect to the nucleotide sequence corresponding to a portion between nucleotide 176 and nucleotide 1762 of the nucleotide sequence shown in SEQ ID NO: 6, and encoding a protein having β-galactoside α2,6-sialyltransferase activity;
(3) a nucleotide sequence corresponding to a portion between nucleotide 3 and nucleotide 1574 of a nucleotide sequence shown in SEQ ID NO: 8; and
(4) a nucleotide sequence comprising a deletion, substitution, and/or addition of one or several nucleotides with respect to the nucleotide sequence corresponding to a portion between nucleotide 3 and nucleotide 1574 of the nucleotide sequence shown in SEQ ID NO: 8, and encoding a protein having β-galactoside α2,6-sialyltransferase activity.

20. A recombinant vector comprising the β-galactoside α2,6-sialyltransferase gene according to claim 18.

21. The recombinant vector accrding to claim 20 which is an expression vector.

22. A transformant transformed with the recombinant vector according to claim 20.

23. A method for producing β-galactoside α2,6-sialyltransferase wherein the transformant of claim 22 is cultured and β-galactoside α2,6-sialyltransferase is collected from the culture.

24. A protein comprising an active domain of β-galactoside α2,6-sialyltransferase having any one of the following amino acid sequences:
(1) an amino acid sequence corresponding to a portion between positions 33 and 529 of the amino acid sequence shown in SEQ ID NO: 5;
(2) an amino acid sequence comprising a deletion, substitution, and/or addition of one or several amino acids with respect to the amino acid sequence corresponding to a portion between positions 33 and 529 of the amino acid sequence shown in SEQ ID NO: 5, and having β-galactoside α2,6-sialyltransferase activity;
(3) an amino acid sequence corresponding to a portion between positions 31 and 524 of the amino acid sequence shown in SEQ ID NO: 7; and
(4) an amino acid sequence comprising a deletion, substitution, and/or addition of one or several amino acids with respect to the amino acid sequence corresponding to a portion between positions 31 and 524 of the amino acid sequence shown in SEQ ID NO: 7, and having β-galactoside α2,6-sialyltransferase activity.

25. An extracellular secretory protein, which comprises a polypeptide portion which is an active domain of the β-galactoside α2,6-sialyltransferase according to claim 16 or 17, and a signal peptide, and has β-galactoside α2,6-sialyltransferase activity.

26. A gene encoding the protein according to claim 24.

27. A recombinant vector comprising the gene according to claim 26.

28. The recombinant vector according to claim 27 which is an expression vector.

29. A transformant transformed with the recombinant vector according to claim 27.

30. A method for producing a protein comprising an active domain of β-galactoside α2,6-sialyltransferase wherein the transformant of claim 29 is cultured and the protein is collected from the culture.


SEQUENCE LISTING (30 sequences)

SEQ ID NO: 1 - protein, 398 aa, Mouse
MRSGGTLFALIGSLMLLLLLRMLWCPADAPARSRLLMEGSREDTSGTSAALKTLWSPTTPVPRTRNSTYLDEKTTQITEKCKDLQYSLNSLSNKTRRYSEDDYLQTITNIQRCPWNRQAEEYDNFRAKLASCCDAIQDFVVSQNNTPVGTNMSYEVESKKHIPIRENIFHMFPVSQPFVDYPYNQCAVVGNGGILNKSLCGAEIDKSDFVFRCNLPPITGSASKDVGSKTNLVTVNPSIITLKYQNLKEKKAQFLEDISTYGDAFLLLPAFSYRANTGISFKVYQTLKESKMRQKVLFFHPRYLRHLALFWRTKGVTAYRLSTGLMIASVAVELCENVKLYGFWPFSKTIEDTPLSHHYYDNMLPKHGFHQMPKEYSQMLQLHMRGILKLQFSKCETA

SEQ ID NO: 2 - DNA, 3166 bp, Mouse
cggagcggcgagtcggtgccgcccgggctgcgcttcgccccggcagctttggcggcgaggacgcccgtggctcaggatgagatcggggggcacgctgttcgccctcataggcagcctgatgctgctgctcctcctgcgtatgctctggtgcccagccgacgcgcctgcccgctccaggctgttgatggagggaagcagagaggacaccagtggtacctcagctgcactgaagacactctggagcccgacaaccccggtaccacgcaccaggaacagcacatatctggatgagaagacaacccaaataacagagaaatgcaaagatctgcaatatagcttgaactctttatctaacaaaacgagacggtactctgaggatgactacctccagaccatcacaaacatacagagatgcccatggaaccggcaagcagaagaatatgacaattttagagcaaaactggcttcctgttgcgatgccattcaagacttcgtggtttcccagaacaacactccagtggggactaacatgagctacgaggtggaaagcaagaaacacatccccattcgagagaacattttccacatgtttccagtgtcgcagccttttgtggactatccctataaccagtgtgcagtggttggtaatgggggaattctcaacaagtctctctgcggagcagaaattgataaatctgacttcgtcttcaggtgtaacctccccccaatcacagggagcgctagtaaagatgttggaagcaaaacaaatcttgtgactgtcaatcccagcattataaccctgaagtaccagaatttgaaggagaagaaagcacagtttttggaggacatctccacctatggagatgcattcctcctcctgccagcattttcctatcgggccaacacaggcatctcttttaaagtctaccaaacactcaaagagtcaaaaatgaggcaaaaggttctcttcttccatcccaggtacctgagacacctcgctcttttctggagaactaaaggggtgactgcataccgcttgtccacaggcttgatgattgcaagtgtcgctgtggaactgtgtgaaaacgtgaagctctacggattctggcctttctctaagactatcgaagacaccccactcagtcaccactactatgataacatgttacctaagcatggtttccaccagatgcctaaagaatacagccaaatgctccagctccatatgagaggaatcctcaaactgcaattcagcaaatgtgaaacggcttaacgtttcttagaaggagaataatttcaggaggtggagtggatgtgtcacagcatctccaaaaagccaatagaagaaggcacagagaaagcatgaattacaaaggcgctctcccacttgtctagaccaaagccacccgcccccactcactttgcagcctccacgagtcactcattctcaccttcaacgttctttctctgagaatagagaccaaaacatcagacttggataagtaaaatgagataatttttcaaatcatcatagaatttgatttgagccagggtctctcagaatgcttccttgttcctatccatgatagccattcccacctttatcagagtggtaatgaaactgtgcaattgtgccaaagaccctttctgaagagaatgtctgaatcatgcgccgagtttttacacacagctcttcctttataaataaatccttcccattctccctcctagtagagtacagaaacaaaatacccttgatgattcaggaagaaaagtcttttttacttagcaatgtgcctgcttctgattcagttcgcttgtgacattaagctgggttggggttttggttggatttggggcgtttcttcacttcttttgtctatattttccttacctttatcagtttgtattcgagcttcctgctttgggattctgcaattctctctcccactgacaggatcaactcaatgacataaagtagttcaaacatccattgcttctcacatgttttatccataaagttactcatctgattttatttaaaatagtgaacatctacttgatatcagacccgaggaccatcctccattggagaatatgaagatattgtcactggcagaaaagcaggtgtgtgccattaattgataagataccacaagcatcatcatgccagttatgaacacagtgctgaaaggatcatagacaggggtggttaaatctgatcccagtagaataaacttcagtgtacctatttcagggaagagttaatttcacaattaaaactagtaaatgaaccaattcttaggcacattaagtggattctgagtaaaagaaagggaacagcaggagaaagctgttcgcttggttctgattacccaaatgagcatgctggaaggaggttgtgaggctacgctaaaacctctgcgtagggagagagtacagtgcatgagtgtggcggcttttgtccacactcgtgaagggtgagtaattcagagccaatcacatcacaaggatggacacacctaactcatcacttcagggggagatgaatgctttcatgagaaattacactcataagctaagcatcagttttgagtaaaatttgagtagatgttaaatatgaacattttatacctcttactaatgtcccaccgacaccttttaatgtaagcacatttatttattaagttacttgacattaaatgcttatgtctgtatattctgttcatccatcgattttcccaaaaagtaagagcataggagatgaggcctacatgccaagaaaactataaattttactctttaattcttacttgagccagcttgttgtttatcaagtgcttttttgaagagacagcaccctgtgaattcttcattctgatacagtgtcaccttgtatttaacatttgtaatgttgtttcaagtttacatctctttcattcttttatagcaaatcaaacgtattagcttcagaaatttatcagaagttcatatataaatattttgcaaagggtaaaaggcttttttgttaaataaaataaaatttattattttcttctgatgaatagaggctcttttatgctgctgctaatgaacctaattagctttaaattatctcctagcaacattggtcacgtttcaatcatgctattagcaaaaaaaaaaa

SEQ ID NO: 3 - protein, 398 aa, Human [sequence omitted]

SEQ ID NO: 4 - DNA, 1500 bp, Human
ggtggcggcggcggcgggagccgcgagtcggggccgcccgggctgtgcttcgccccggcagcagcggtggcggcggcgcctgtggctcaggatgcggccggggggcgcactgctcgccctgctcgccagcctgctgctgctgctgctgctgcgcctgctctggtgcccggcagacgcgcccggccgcgccaggattctggtggaggaaagcagggaggccacccacggcacccccgcagcgctgaggacgctccggagcccggcgaccgcggtaccgcgcgccactaacagcacatatctgaatgagaagtcgctccaactgacggagaaatgcaaaaatctgcaatatggcattgagtctttctctaacaaaacgaaagggtattcagagaacgactaccttcagattatcacagatatacagagttgtccatggaaacggcaagcagaagaatatgcaaattttagagccaaacttgcttcctgctgtgatgctgttcaaaactttgttgtttctcagaataacactccagttgggactaatatgagttacgaggtggaaagcaaaaaagaaatcccaattaagaagaacatttttcatatgtttccagtgtcccagccttttgtggactacccttataatcagtgtgcagtggtcggaaatgggggaattctgaataagtctctctgtggaactgaaatagataaatccgacttcgtttttaggtgtaacctacccccaaccacaggagatgttagtaaagatgttggcagtaaaacaaatcttgtgactataaatccaagcatcataactctgaaatatgggaacttaaaggaaaaaaaagccctatttttggaggacattgcaacctatggagatgcatttttttttctgccagcattttccttcagggccaacacgggtacctctttcaaagtatactacacgctcgaagagtctaaagcaagacaaaaggttctatttttccatcccaagtacctgaaagatctggcccttttctggagaactaaaggtgtgactgcataccgcttgtccaccggcttgatgatcacaagtgttgcagtggaactgtgtaaaaatgtgaagctgtatggattctggcccttctctaaaactgtagaagacatacctgtcagccatcactattatgacaacaagctacctaaacatggtttccatcagatgcccaaagaatacagccagatcctccaacttcacatgaaaggaatcctcaaactgcaatttagcaaatgtgaagtcgcctaaacaaagtatcttaaaatgggaataattttaatataatgcagtaggtgattaacaatgtctccaaacaccaaaggaggtggctaaagagtattttgagatgagccccaaaatttggtttgaccaaagcttccccactcattttgcaatgatggcaagtcattcaatccttctcatcttcattttttctccttataacatggacaccatatctg

SEQ ID NO: 5 - protein, 529 aa, Human
MKPHLKQWRQRMLFGIFAWGLLFLLIFIYFTDSNPAEPVPSSLSFLETRRLLPVQGKQRAIMGAAHEPSPPGGLDARQALPRAHPAGSFHAGPGDLQKWAQSQDGFEHKEFFSSQVGRKSQSAFYPEDDDYFFAAGQPGWHSHTQGTLGFPSPGEPGPREGAFPAAQVQRRRVKKRHRRQRRSHVLEEGDDGDRLYSSMSRAFLYRLWKGNVSSKMLNPRLQKAMKDYLTANKHGVRFRGKREAGLSRAQLLCQLRSRARVRTLDGTEAPFSALGWRRLVPAVPLSQLHPRGLRSCAVVMSAGAILNSSLGEEIDSHDAVLRFNSAPTRGYEKDVGNKTTIRIINSQILTNPSHHFIDSSLYKDVILVAWDPAPYSANLNLWYKKPDYNLFTPYIQHRQRNPNQPFYILHPKFIWQLWDIIQENTKEKIQPNPPSSGFIGILIMMSMCREVHVYEYIPSVRQTELCHYHELYYDAACTLGAYHPLLYEKLLVQRLNMGTQGDLHRKGKVVLPGFQAVHCPAPSPVIPHS

SEQ ID NO: 6 - DNA, 1800 bp, Human
ggcgccgggactccctcctggccgcccacagcctgtgcgcattcctgcattcctgccgccgcccgggacccgagcccccggaggtgtccaggcgcggtgccaggcgggtactgtgcaggttcattctgccacccatctgcattaagacacaaggtgctgaccgcagagacctgccatgaaaccacacttgaagcaatggagacaacgaatgcttttcggaatattcgcttgggggctcctctttttgctgattttcatctacttcaccgacagcaaccccgctgagcctgtacccagctccctctccttcctggagaccaggaggctcctgccggtgcaggggaagcagcgggccatcatgggcgccgcacatgagccctccccgcctgggggcctggacgcacgccaggcgctgccccgcgcccacccagccggttcctttcatgcggggcctggagacctgcagaaatgggcccagtcccaagatgggtttgaacataaagagtttttttcatcccaggtggggagaaaatctcaaagtgctttctacccggaggatgacgactacttttttgctgctggtcagccagggtggcacagccacactcaggggacattgggattcccttcccccggggagccaggcccacgggagggggcttttccggctgcacaggtccagaggaggcgggtgaagaagaggcaccggaggcagagaaggagccacgtgttggaggagggcgacgacggcgacaggctgtactcctccatgtccagggccttcctgtaccggctctggaaggggaacgtctcttccaaaatgctgaacccgcgcctgcagaaggcgatgaaggattacctgaccgccaacaagcacggggtgcgcttccgcgggaagcgggaggccgggctgagcagggcacagctgctgtgccagctgcggagccgcgcgcgcgtgcggacgctggacggcaccgaggcgcccttttctgcgctgggctggcggcgcctggtgcccgccgtgcccctgagccagctgcacccccgcggcctgcgcagctgcgctgtcgtcatgtctgcaggcgcaatcctcaactcttccttgggcgaggaaatagattctcatgatgcggttttgagatttaactctgctcctacacgtggttatgagaaagatgttgggaataaaaccaccatacgcatcattaattcgcagattctgaccaaccccagccatcacttcattgacagttcactgtataaagacgtcattttggtggcctgggaccctgccccatattccgcaaatcttaacctgtggtacaaaaaaccggattacaacctgttcactccatatattcagcatcgtcagagaaacccaaatcagccattttacattcttcatcctaaatttatatggcagctctgggatattatccaggagaacactaaagagaagattcaaccaaacccaccatcttctggtttcattggaatcctcatcatgatgtccatgtgcagagaggtgcacgtgtatgaatatatcccatccgtgcggcagacggagctgtgccactaccacgagctgtactacgacgcagcctgcaccctcggggcgtaccacccactactctatgagaagctcctggtgcagcgcctgaacatgggcacgcagggggatttgcatcgcaagggcaaggtggttcttcctggcttccaggcggtgcactgccctgcaccaagtccagtcattccacactcttaaaaagggtttcttgggaatcaatgtgcaatggtaca

SEQ ID NO: 7 - protein, 524 aa, Mouse
MKPHLKQWRQRMLFGIFVWGLLFLAIFIYFTNSNPAAPMPSSFSFLESRGLLPLQGKQRVIMGALQEPSLPRSLDASKVLLDSHPENPFHPWPGDPQKWDQAPNGFDNGDEFFTSQVGRKSQSAFYPEEDSYFFVADQPELYHHRQGALELPSPGETSWRSGPVQPKQKLLHPRRGSLPEEAYDSDMLSASMSRAFLYRLWKGAVSSKMLNPRLQKAMRYYMSFNKHGVRFRRRGRREATRTGPELLCEMRRRVRVRTLDGREAPFSGLGWRPLVPGVPLSQLHPRGLSSCAVVMSAGAILNSSLGEEIDSHDAVLRFNSAPTRGYEKDVGNKTTVRIINSQILANPSHHFIDSALYKDVILVAWDPAPYSANLNLWYKKPDYNLFTPYIQHRRKYPTQPFYILHPKFIWQLWDIIQENTREKIQPNPPSSGFIGILIMMSMCKEVHVYEYIPSVRQTELCHYHELYYDAACTLGAYHPLLYEKLLVQRLNTGTQADLHHKGKVVLPGFQTLRCPVTSPNNTHS

SEQ ID NO: 8 - DNA, 1611 bp, Mouse
caatgaaaccacacttgaagcaatggcgacaacgaatgctctttggaatatttgtttgggggctcctctttttggcaattttcatctacttcaccaacagcaatcctgcggcacctatgcccagctccttttccttcctggagagccgtgggctcctgcctctacagggcaagcagcgggtcatcatgggcgctttgcaggaaccctctttgcccagaagtttggatgcaagcaaagtgcttctggacagccaccctgagaaccctttccacccttggcctggggacccacagaaatgggatcaggccccaaatggctttgacaatggggatgagttttttacatcccaggttgggaggaaatcacaaagcgctttctatcccgaggaagatagctatttttttgttgcggatcagcctgagttgtaccaccacaggcagggtgcactggagctgccatctccaggggagacatcatggcgatcaggacctgttcagcccaagcagaagctgcttcacccaaggcgaggcagcttgcctgaggaagcctatgacagcgacatgctgtcagcctccatgtcgagagccttcctgtaccggctctggaagggggccgtgtcctctaagatgttgaacccgcgcctgcagaaggccatgcgttactacatgtccttcaacaagcatggtgtgcgcttccgcaggcggggtcggcgtgaagctacacgtacagggccggagctgctgtgtgagatgcgcagacgtgtgcgtgtgcgcacgttggacggcagagaggcgcccttctcggggctgggctggcggcctctggtaccaggtgtacctctgagccagttgcacccgcgcggtctgagcagctgcgcagttgtcatgtctgccggtgccatcctgaactcctccttgggggaggaaatcgattctcatgatgcagttttgagatttaactctgcccctacccgtggctacgagaaagatgtcggaaataaaaccacagtacgcatcattaattctcagattctggccaaccccagccatcacttcattgacagtgctttatataaagatgttatcctggtagcctgggatcctgctccttattctgccaatcttaacctgtggtataagaagccagattacaaccttttcactccatatatccagcatcgccggaaatacccgactcagccattttacattcttcaccccaagttcatatggcagctttgggacattatccaggagaatacaagggagaagatacagcccaacccaccatcttctggttttattggaatcctcatcatgatgtccatgtgtaaagaggtgcacgtgtatgagtacatcccatctgttcgacagacagagctttgccactaccatgagctgtactacgacgcagcctgcaccttgggggcctaccacccactgctctatgaaaagctactggtgcagcgccttaacacaggcacccaggcagacttgcatcacaagggcaaggtagtcttgccaggcttccagacccttcggtgtccagtaaccagccccaacaatacacattcttaaaatggaactcttgggaactgatgtgcaataaggt

SEQ ID NO: 9 - DNA, 20 bp, Artificial Sequence
  Synthetic DNA
cttttctggagaactaaagg

SEQ ID NO: 10 - DNA, 20 bp, Artificial Sequence
  Synthetic DNA
aattgcagtttgaggattcc

SEQ ID NO: 11 - DNA, 20 bp, Artificial Sequence
  Synthetic DNA
tggctcaggatgagatcggg

SEQ ID NO: 12 - DNA, 22 bp, Artificial Sequence
  Synthetic DNA
tactagcgctccctgtgattgg

SEQ ID NO: 13 - DNA, 30 bp, Artificial Sequence
  Synthetic DNA
tgctctcgagcccagccgacgcgcctgccc

SEQ ID NO: 14 - DNA, 30 bp, Artificial Sequence
  Synthetic DNA
tattctcgagctaagaaacgttaagccgtt

SEQ ID NO: 15 - DNA, 30 bp, Artificial Sequence
  Synthetic DNA
caattgacatatctgaatgagaagtcgctc

SEQ ID NO: 16 - DNA, 22 bp, Artificial Sequence
  Synthetic DNA
tactaacatctcctgtggttgg

SEQ ID NO: 17 - DNA, 20 bp, Artificial Sequence
  Synthetic DNA
ccagtgtcccagccttttgt

SEQ ID NO: 18 - DNA, 20 bp, Artificial Sequence
  Synthetic DNA
tgagtggggaagctttggtc

SEQ ID NO: 19 - DNA, 30 bp, Artificial Sequence
  Synthetic DNA
gacaatggggatgagttttttacatcccag

SEQ ID NO: 20 - DNA, 29 bp, Artificial Sequence
  Synthetic DNA
cgatttcctcccccaaggaggagttcagg

SEQ ID NO: 21 - DNA, 30 bp, Artificial Sequence
  Synthetic DNA
acgttggacggcagagaggcgcccttctcg

SEQ ID NO: 22 - DNA, 30 bp, Artificial Sequence
  Synthetic DNA
accttattgcacatcagttcccaagagttc

SEQ ID NO: 23 - DNA, 30 bp, Artificial Sequence
  Synthetic DNA
caatgaaaccacacttgaagcaatggcgac

SEQ ID NO: 24 - DNA, 30 bp, Artificial Sequence
  Synthetic DNA
cgcaacaaaaaaatagctatcttcctcggg

SEQ ID NO: 25 - DNA, 30 bp, Artificial Sequence
  Synthetic DNA
tcatctacttcacctcgagcaaccccgctg

SEQ ID NO: 26 - DNA, 30 bp, Artificial Sequence
  Synthetic DNA
catccaattgaccaacagcaatcctgcggc

SEQ ID NO: 27 - DNA, 23 bp, Artificial Sequence
  Synthetic DNA
ttatgattcacaccaacctgaag

SEQ ID NO: 28 - DNA, 23 bp, Artificial Sequence
  Synthetic DNA
ctttgtacttgttcatgcttagg

SEQ ID NO: 29 - DNA, 23 bp, Artificial Sequence
  Synthetic DNA
agacgtcattttggtggcctggg

SEQ ID NO: 30 - DNA, 21 bp, Artificial Sequence
  Synthetic DNA
ttaagagtgtggaatgactgg